(19) United States
(12) Patent Application Publication
(10) Pub. No.: US 2010/0055078 A1
(43) Pub. Date: Mar. 4, 2010

(54) METHODS OF MAKING A TRANSPLANTABLE BONE REPAIR SYSTEM USING MULTIPOTENT STEM CELLS

(75) Inventor: Millie Hughes-Fulford, Mill Valley, CA (US)

Correspondence Address: KLARQUIST SPARKMAN, LLP, 121 SW SALMON STREET, SUITE 1600, PORTLAND, OR, 97204, US

(73) Assignees: The Government of the U.S.A, as represented by the Department of Veterans Affairs; of Veterans Affairs

(21) Appl. No.: 12/541,401

(22) Filed: Aug. 14, 2009

Related U.S. Application Data
(60) Provisional application No. 61/089,425, filed on Aug. 15, 2008.

(51) Int. Cl. A61K 35/12; C12N 5/02

(52) U.S. Cl. 424/93.7; 435/384

(57) ABSTRACT

Methods are provided herein for making a biocompatible transplantable bone repair device. In some examples, the method includes culturing multipotent stem cells with a first medium containing a fibroblast growth factor (FGF), such as fibroblast growth factor-2 (FGF-2), in the substantial absence of dexamethasone, seeding a biologically compatible substrate including extracellular matrix (ECM) with the multipotent stem cells cultured with FGF, and inducing the cells to differentiate to osteoblast cells by culturing with a second medium containing a differentiation factor (such as bone morphogenetic protein, such as bone morphogenetic protein-2 (BMP-2)), in the substantial absence of dexamethasone. Also provided are methods for treating a bone defect by surgically implanting the biocompatible transplantable bone repair device at the site of a bone defect.

### CROSS REFERENCE TO RELATED APPLICATION

This application claims the benefit of U.S. Provisional Application No. 61/089,425, filed Aug. 15, 2008, which is incorporated herein in its entirety.

### ACKNOWLEDGMENT OF GOVERNMENT SUPPORT

This invention was made with government support under VA Merit Review awarded by The Department of Veterans Affairs and grant W81WH-07-0427 awarded by the United States Army. The government has certain rights in the invention.

### FIELD

This application relates to the field of bone repair, specifically to the use of multipotent stem cells to make a biocompatible transplantable bone repair system for treating bone defects.

### BACKGROUND

Traumatic bone injury greatly increases morbidity and mortality and significantly delays or prevents return to normal life following injury. Full recovery from significant bone injuries is limited and causes substantial distress and economic loss. Bone injury becomes critical when there is a lack of sufficient remaining bone, stem cells, and an inability to release osteogenic growth factors. Many times this is accompanied by an insufficient infrastructure (scaffold) upon which to rebuild bone. A contributing factor to the inability to achieve critical defect/non-union fracture repair is a deficiency in angiogenesis. Ischemic osteonecrosis is also a common complication in trauma, often leading to infection and amputation. Therefore, a lack of sufficient growth factors, scaffold, stem cells, and angiogenesis contribute to inability of the critical defect to heal properly. There is a need for improved methods for treating critical bone defects to reduce morbidity and mortality due to these injuries.

### SUMMARY

Disclosed herein are methods for making a biocompatible transplantable bone repair system. The methods include culturing multipotent stem cells with a first medium containing a fibroblast growth factor (FGF), such as fibroblast growth factor-2 (FGF-2), in the substantial absence of dexamethasone, seeding a biologically compatible substrate including extracellular matrix (ECM) with the multipotent stem cells cultured with FGF, and inducing the cells to differentiate to osteogenic cells by culturing with a second medium containing a differentiation factor (such as bone morphogenetic protein (BMP), for example, bone morphogenetic protein-2 (BMP-2)), in the substantial absence of dexamethasone.

Also disclosed are methods for treating a bone defect. The methods include culturing multipotent stem cells with a first medium containing an FGF, such as FGF-2, in the substantial absence of dexamethasone, seeding a biologically compatible substrate including ECM with the multipotent stem cells cultured with FGF, inducing the cells to differentiate to osteogenic cells by culturing with a second medium containing a differentiation factor (such as a BMP, for example, BMP-2), in the substantial absence of dexamethasone, and surgically implanting the biocompatible substrate including the osteogenic cells at the site of a bone defect.

In some examples, the ECM includes at least one ECM component, such as collagen, fibrinogen, laminin, fibronectin, fibrin, hyaluronic acid, elastin, or entactin. In a particular example, the ECM includes fibrinogen.

In additional examples, the biologically compatible substrate includes materials such as collagen, polymers (for example, nylon or polyester), tricalcium phosphate, hydroxyapatite, or other biocompatible scaffolds. In a particular example, the biocompatible substrate is a collagen sponge or a nylon strip.

In some examples, the multipotent stem cells are post-natal stem cells, such as cells obtained from bone marrow or adipose tissues, for example, mesenchymal stem cells or adipose-derived stem cells.

The foregoing and other features of the disclosure will become more apparent from the following detailed description, which proceeds with reference to the accompanying figures.

### BRIEF DESCRIPTION OF THE DRAWINGS

FIG. 1 is a bar graph showing DNA synthesis in untreated wounded cell defect controls and in defects treated with FGF-2 (0.2 ng/ml), transforming growth factor β (TGFβ) (2 ng/ml), prostaglandin E₂ (PGE₂) (5 μg/ml), insulin-like growth factor-1 (IGF-1) (20 ng/ml), and platelet derived growth factor (PDGF) (3 ng/ml) assessed by [³H]-thymidine incorporation during 24 hour treatment of MC3T3-E1 osteoblasts. Each bar represents the mean value of 6 determinations±standard deviation. * p<0.05,** p<0.01 and *** p<0.0001

FIG. 2 is a series of fluorescent micrographs of MC3T3-E1 cell monolayers that were wounded and treated with or without growth factors. Cell morphology was imaged using F-actin specific stain, rhodamine phalloidin. Control, untreated control cells; TGFβ, cells treated with TGFβ (2 ng/ml); PGE₂, cells treated with 5 μg/ml PGE₂; FGF-2, cells treated with 0.2 ng/ml FGF-2. Arrows mark the site of the initial wound before re-growth.

FIG. 3 is a bar graph showing the relative cell number of human mesenchymal stem cells (MSCs) treated with increasing amounts of FGF-2 or with 10% fetal bovine serum (FBS) for 48 hours. Each bar represent mean±SD (n=8); * p<0.05; ** p<0.01; *** p<0.0001 relative to untreated control with two-tailed student t test.

FIG. 4 is a series of bar graphs showing qRT-PCR analysis of gene expression in MC3T3-E1 cells over 24 hours of treatment with FGF-2. Each bar represents mean±SD in triplicate independent biological samples with each time point corrected to cyclophilin (* p<0.05; ** p<0.01 with two-tail student t-test compared to 0 hour of each gene).

FIG. 5 is a series of bar graphs showing qRT-PCR analysis of gene expression over 24 hours of treatment of MC3T3-E1 cells with FGF-2. Each bar represents mean±SD in triplicate independent biological samples with each time point corrected to cyclophilin (* p<0.05; ** p<0.01 with two-tail student t-test compared to 0 hour of each gene).

FIG. 6 is a bar graph showing the effect of twenty-four hours of treatment with FGF-2 or BMP-2 on fold increase in relative abundance of mineralization-related gene expression. Each bar represents mean±SD in triplicate independent biological samples with each time point corrected to cyclophilin. * p<0.05; ** p<0.01 with two-tail student t-test compared to 0 hour of each gene. * p<0.05; ** p<0.01; *** p<0.0001 FGF-2 vs. BMP-2 treatment. NT=not treated.

FIG. 7 is a bar graph showing cell number in MC3T3-E1 cells cultured for 48 hours with or without FGF-2 and ECM proteins (Ctrl, no matrix; MG, MATRIGEL®; FN, fibronectin; FNLP; fibronectin like protein; HA, hyaluronic acid; LM, laminin; Col I, collagen type I). Each bar represents mean±SD (n=5); * p<0.05; ** p<0.01 relative to untreated control with two tail student t test. NT=not treated.

FIG. 8 is a series of epi-fluorescence photomicrographs showing hMSCs 4 days after seeding on collagen sponges pre-treated with the indicated growth factor/ECM mixtures. The cells were stained with rhodamine phalloidin to show F-actin fibers. HAHp, 0.3% w/w heparin with 1% hyaluronic acid; FGF-2, 100 ng/ml FGF-2.

FIG. 9 is a series of photomicrographs showing mouse MSCs 6 days after seeding on GELFOAM® sponges pre-treated with the indicated growth factor/ECM mixtures. FGF-2, 100 ng/ml; Fibrinogen, 1 mg/ml; HAHp, 10 mg/ml hyaluronic acid+3 mg/ml heparin. Arrows identify cells and cell clusters attached to the substrate.

FIG. 10 is a series of photomicrographs showing MC3T3-E1 cells 4 days after seeding on nylon strips pre-treated with the indicated growth factor/ECM mixtures. FGF-2, 50 ng/ml; Fibrinogen, 0.1 mg/ml, HAHp, 1 mg/ml hyaluronic acid+3 mg/ml heparin. Arrows indicate attachment of cells to the substrate.

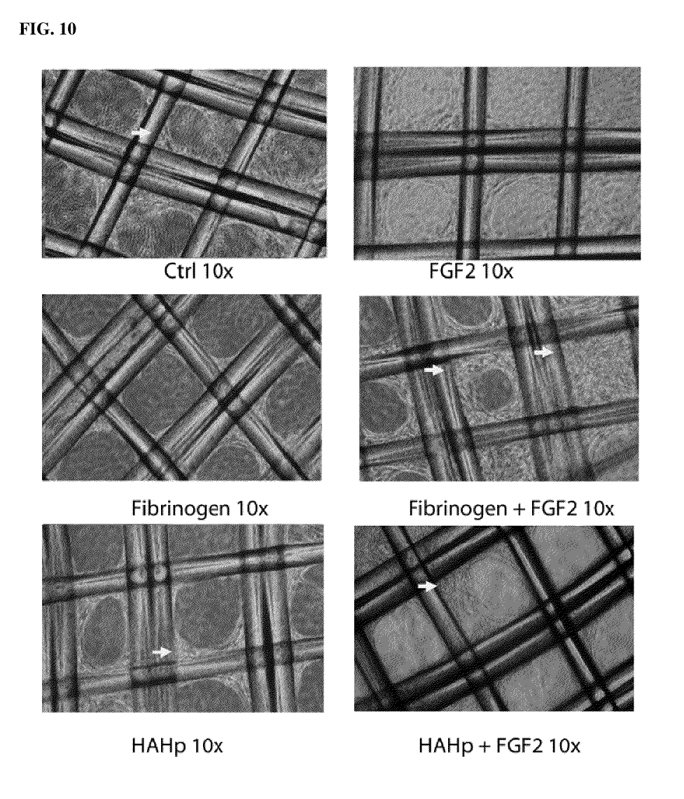

FIG. 11 is a series of photomicrographs showing confluent MC3T3-E1 osteoblast cells treated with the indicated media for two days and stained with 2% Alizarin Red. Arrows indicate areas of mineralization (red staining).

### DETAILED DESCRIPTION

### I. Terms and Abbreviations

Unless otherwise explained, all technical and scientific terms used herein have the same meaning as commonly understood by one of ordinary skill in the art to which this disclosure belongs. The singular terms “a,” “an,” and “the” include plural referents unless the context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” All publications, patent applications, patents, and other references mentioned herein are incorporated by reference in their entirety for all purposes. In case of conflict, the present specification, including explanations of terms, will control. In addition, the materials, methods, and examples are illustrative only and not intended to be limiting. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).
ASC: adipose-derived stem cell
BMP: bone morphogenetic protein
BMP-2: bone morphogenetic protein-2
COL1A1: collagen type I
ECM: extracellular matrix
EN: entactin
FGF: fibroblast growth factor
FGF-2: fibroblast growth factor-2 (basic fibroblast growth factor)
FN: fibronectin
GAG: glycosaminoglycan
HA: hyaluronic acid
IGF-1: insulin-like growth factor-1
IGF-2: insulin-like growth factor-2
MSC: mesenchymal stem cell
OC: osteocalcin
PDGF: platelet-derived growth factor
qRT-PCR: quantitative real-time reverse-transcriptase PCR
RWV: rotating wall vessel
TGFβ: transforming growth factor β
ACT: microcomputed tomography

Antibody: A protein (or protein complex) that includes one or more polypeptides substantially encoded by immunoglobulin genes or fragments of immunoglobulin genes. The recognized immunoglobulin genes include the kappa, lambda, alpha, gamma, delta, epsilon, and mu constant region genes, as well as the myriad of immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The basic immunoglobulin (antibody) structural unit is generally a tetramer. Each tetramer is composed of two identical pairs of polypeptide chains, each pair having one “light” (about 25 kDa) and one “heavy” (about 50-70 kDa) chain. The N-terminus of each chain defines a variable region of about 100 to 110 or more amino acids primarily responsible for antigen recognition. The terms “variable light chain” (V_{L}) and “variable heavy chain” (V_{H}) refer, respectively, to these light and heavy chains.

As used herein, the term “antibodies” includes intact immunoglobulins as well as a number of well-characterized fragments. For instance, Fabs, Fvs, and single-chain Fvs (SCFvs) that bind to target protein (or epitope within a protein or fusion protein) would also be specific binding agents for that protein (or epitope). These antibody fragments are defined as follows: (1) Fab, the fragment which contains a monovalent antigen-binding fragment of an antibody molecule produced by digestion of whole antibody with the enzyme papain to yield an intact light chain and a portion of one heavy chain; (2) Fab′, the fragment of an antibody molecule obtained by treating whole antibody with pepsin, followed by reduction, to yield an intact light chain and a portion of the heavy chain; two Fab′ fragments are obtained per antibody molecule; (3) (Fab′)₂, the fragment of the antibody obtained by treating whole antibody with the enzyme pepsin without subsequent reduction; (4) F(ab′)₂, a dimer of two Fab′ fragments held together by two disulfide bonds; (5) Fv, a genetically engineered fragment containing the variable region of the light chain and the variable region of the heavy chain expressed as two chains; and (6) single chain antibody, a genetically engineered molecule containing the variable region of the light chain, the variable region of the heavy chain, linked by a suitable polypeptide linker as a genetically fused single chain molecule. Methods of making these fragments are routine (see, for example, Harlow and Lane, Using Antibodies: A Laboratory Manual, CSHL, New York, 1999).

Antibodies for use in the methods and devices of this disclosure can be monoclonal or polyclonal. Merely by way of example, monoclonal antibodies can be prepared from murine hybridomas according to the classical method of Kohler and Milstein (Nature 256:495-97, 1975) or derivative methods thereof. Detailed procedures for monoclonal antibody production are described in Harlow and Lane, Using Antibodies: A Laboratory Manual, CSHL, New York, 1999.

Biocompatible: The property of a biomaterial or device having the ability to perform its desired function (for example, with respect to a medical therapy), without eliciting any undesirable local or systemic effects in a subject. A biocompatible material or device ideally also generates a beneficial effect or cellular or tissue response. In some examples, biocompatible refers to a material or device that is enzymatically or chemically degraded in vivo into simpler chemical species (“biodegradable”). A biocompatible material, device, or system includes synthetic or natural material used to replace part of a living system or to function in intimate contact with living tissue. The terms “biocompatible” and “biologically compatible” are used interchangeably herein.

Bone defect: Includes any disease, defect, or disorder which affects bone strength, function, and/or integrity, such as those resulting from injury, or a defect brought about during the course of surgery, infection, malignancy, or developmental malformation. Examples of bone defects include, but are not limited to, fractures (such as a critical defect or non-union fracture), dental or facial defects (such as cleft palate or facial or dental injuries or malformations). Other examples of bone defects include damage to bones resulting from diseases of bone fragility, such as osteoporosis, and malignancies and/or cancers of the bone such as a sarcoma, such as osteosarcoma.

Bone morphogenetic protein (BMP): A family of proteins, identified originally in extracts of demineralized bone that were capable of inducing bone formation at ectopic sites. BMPs are found in minute amounts in bone material (approximately 1 μg/kg dry weight of bone). Most members of this family (with the exception of BMP-1) belong to the transforming growth factor (TGF)-β family of proteins.

BMPs can be isolated from demineralized bones and osteosarcoma cells. They have been shown also to be expressed in a variety of epithelial and mesenchymal tissues in the embryo. BMPs are proteins which act to induce the differentiation of mesenchymal-type cells into chondrocytes and osteoblasts before initiating bone formation. They promote the differentiation of cartilage- and bone-forming cells near sites of fractures but also at ectopic locations. Some of the proteins induce the synthesis of alkaline phosphatase and collagen in osteoblasts. Some BMPs act directly on osteoblasts and promote their maturation while at the same time suppressing myogenous differentiation. Other BMPs promote the conversion of typical fibroblasts into chondrocytes and are capable also of inducing the expression of an osteoblast phenotype in non-osteogenic cell types. BMPs include BMP-1 to BMP-15, such as BMP-2. BMP-2, BMP-4 and BMP-7 have been shown to promote bone formation.

BMP sequences are publicly available. For example, GENBANK® Accession numbers NP_{—}031579 and NP_{—}001191 disclose exemplary mouse and human BMP-2 amino acid sequences, respectively (sequences associated with GENBANK® Accession Numbers as of Aug. 15, 2008 are herein incorporated by reference). In certain examples, BMP-2 has at least 80% sequence identity, for example at least 85%, 90%, 95%, or 98% sequence identity to a publicly available BMP-2 sequence.

Cell culture: Growth of a population of cells in a defined set of conditions (such as culture medium, temperature, and/or time of culture). In some examples, a cell culture includes a substantially pure culture (for example, isolated mesenchymal stem cells or adipose-derived stem cells). In additional examples a cell culture includes a mixed culture, such as co-culture of two or more types of cells (for example a culture of bone marrow cells, including one or more of fibroblasts, macrophages, mesenchymal stem cells, and hematopoietic cells), cells at different stages of differentiation (for example, a mixture of multipotent stem cells and differentiated cells) or a combination thereof. In further examples, a cell culture includes cells grown in contact with one or more biocompatible substrates, such as a sponge, strip, scaffold, or gel (such as a collagen sponge or nylon strip).

Conservative substitution: A substitution of an amino acid residue for another amino acid residue having similar biochemical properties. Typically, conservative substitutions have little to no impact on the biological activity of a resulting polypeptide (such as FGF-2 or BMP-2). In a particular example, a conservative substitution is an amino acid substitution in a peptide that does not substantially affect the biological function of the peptide. A peptide can include one or more amino acid substitutions, for example 2-10 conservative substitutions, 2-5 conservative substitutions, 4-9 conservative substitutions, such as 2 or 5. Examples of conservative substitutions are shown below.

| Original | Conservative |
|---|---|
| Residue | Substitutions |
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain.

The substitutions which in general are expected to produce the greatest changes in protein properties will be non-conservative, for instance changes in which (a) a hydrophilic residue, for example, seryl or threonyl, is substituted for (or by) a hydrophobic residue, for example, leucyl, isoleucyl, phenylalanyl, valyl or alanyl; (b) a cysteine or proline is substituted for (or by) any other residue; (c) a residue having an electropositive side chain, for example, lysyl, arginyl, or histadyl, is substituted for (or by) an electronegative residue, for example, glutamyl or aspartyl; or (d) a residue having a bulky side chain, for example, phenylalanine, is substituted for (or by) one not having a side chain, for example, glycine.

Degenerate variant: A polynucleotide encoding a protein (such as FGF-2, BMP-2, or other proteins disclosed herein) that includes a sequence that is degenerate as a result of the genetic code. There are 20 natural amino acids, most of which are specified by more than one codon. Therefore, all degenerate nucleotide sequences are included as long as the amino acid sequence of the polypeptide encoded by the nucleotide sequence is unchanged.

Dexamethasone: A synthetic glucocorticoid hormone that has anti-inflammatory and immunosuppressant activities. Dexamethasone may be used in some situations (such as in cell culture systems) to promote differentiation of cells (such as stem cells) to osteogenic cells. In examples provided herein, cell culture medium utilized for the growth and/or differentiation of multipotent stem cells (such as MSCs or ASCs) includes FGF-2 or BMP-2 in the substantial absence of dexamethasone, such as medium which does not contain added or exogenous dexamethasone, medium that contains less than about 10 nM dexamethasone (such as less than about 5 nM, less than about 1 nM, or less than about 0.1 nM dexamethasone), or medium that does not contain detectable levels of dexamethasone.

Differentiate: A change in the characteristics of a cell such that a less specialized cell becomes more specialized. Differentiation may cause changes in the size, shape, polarity, gene expression, metabolic activity, and responsiveness to signals of a cell. In a particular example, a multipotent stem cell differentiates to an osteogenic cell when the cell exhibits at least one marker of mineralization, such as an increase in expression or activity of a mineralization gene or protein or an increase in calcium secretion.

DNA (deoxyribonucleic acid): DNA is a long chain polymer which comprises the genetic material of most living organisms (some viruses have genes comprising ribonucleic acid (RNA)). The repeating units in DNA polymers are four different nucleotides, each of which comprises one of the four bases, adenine (A), guanine (G), cytosine (C), and thymine (T) bound to a deoxyribose sugar to which a phosphate group is attached. Triplets of nucleotides (referred to as codons) code for each amino acid in a polypeptide, or for a stop signal. The term codon is also used for the corresponding (and complementary) sequences of three nucleotides in the mRNA into which the DNA sequence is transcribed.

Unless otherwise specified, any reference to a DNA molecule is intended to include the reverse complement of that DNA molecule. Except where single-strandedness is required by the text herein, DNA molecules, though written to depict only a single strand, encompass both strands of a double-stranded DNA molecule. Thus, a reference to the nucleic acid molecule that encodes a specific protein, or a fragment thereof, encompasses both the sense strand and its reverse complement. For instance, it is appropriate to generate probes or primers from the reverse complement sequence of the disclosed nucleic acid molecules.

Extracellular matrix (ECM): A complex mixture of proteins, proteoglycans, minerals, and other components that surrounds cells and provides structural support, as well as influencing cell survival, development, migration, proliferation, shape, and function. In some examples, ECM includes one or more ECM components, such as proteins (for example, collagen (for example, collagen Type I or collagen Type IV), elastin, laminin, fibronectin, fibrin, fibrinogen, and entactin) or polysaccharides (for example, chondroitin sulfate, dermatan sulfate, heparan sulfate, keratan sulfate, and hyaluronic acid), such as combinations of two or more thereof. As used herein, ECM refers to one or more components of the ECM that is typically found around mammalian cells. In a particular example, ECM includes fibrinogen or fibronectin.

Fibroblast growth factor (FGF): A large multigene family of growth factors that is a pleiotropic regulator of the proliferation, differentiation, migration, and survival in a variety of cell types (see Bikfalvi et al., Endocrine Rev. 18:26-45, 1997). The proteins in this family are 16-18 kDa proteins controlling normal growth and differentiation of mesenchymal, epithelial, and neuroectodermal cell types.

Two main groups of FGFs are known. One type of FGF was isolated initially from brain tissue and identified by its ability to enhance proliferation of murine fibroblasts. Due to its basic pI the factor was named basic FGF (bFGF) or FGF-2 (see below). This factor is the prototype of the FGF family. Another factor, also isolated initially from brain tissues, has the ability to enhance proliferation of myoblasts. This factor is termed acidic FGF (aFGF, also known as FGF-1). Other proteins in the FGF family are int-2 (FGF-3), FGF-4, FGF-5, FGF-6, K-FGF (FGF-7), and FGF-8. All of these factors are products of different genes. Some FGFs are not secreted (FGF-2) while others (FGF-3, FGF-4, FGF-5 and FGF-6) have a signal sequence. Presently there are 23 factors identified as an FGF (numbered FGF-1 to FGF-23).

Basic fibroblast growth factor (bFGF or FGF-2) is a potent stimulator of angiogenesis (see D'Amore and Smith, Growth Factors 8:61-75, 1993) and hematopoiesis in vivo (see Allouche and Bikfalvi, Prog. Growth Factor Res. 6:35-48, 1995). FGF-2 is also involved in organogenesis (Martin, Genes Dev. 12:1571-1586, 1998), vascularization (see Friesel and Maciag, FASEB J. 9:919-925, 1995), and wound healing (see Ortega et al., Proc. Natl. Acad. Sci. USA 95:5672-5677, 1998), and plays an important role in the differentiation and/or function of various organs, including the nervous system (see Ortega et al., Proc. Natl. Acad. Sci. USA 95:5672-5677, 1998) and the skeleton (see Montero et al., J. Clin. Invest. 105:1085-1093, 2000). Because of its angiogenic and anabolic properties, FGF-2 has been shown to be involved in wound healing.

FGF-2, and other FGFs, can be made as described in U.S. Pat. No. 5,155,214. The recombinant FGF-2, and other FGFs, can be purified to pharmaceutical quality (98% or greater purity). FGF-2 sequences are publicly available. For example, GENBANK® Accession numbers NP_{—}032032 and NP_{—}001997 disclose exemplary mouse and human FGF-2 amino acid sequences, respectively (sequences associated with GENBANK® Accession Numbers as of Aug. 15, 2008 are herein incorporated by reference). In certain examples, FGF-2 has at least 80% sequence identity, for example at least 85%, 90%, 95%, or 98% sequence identity to a publicly available FGF-2 sequence.

Fracture: A medical condition in which a bone is cracked or broken; a break in the continuity of a bone. Fractures may be classified as closed or open. A closed fracture is one in which the skin is intact; an open (or compound) fracture is one in which the bone is in contact with the air (such as piercing the skin or due to severe tissue injury). Fractures are also classified as simple or multi-fragmentary. A simple fracture occurs along only one line (such as splitting a bone into two pieces), while a multi-fragmentary fracture splits a bone into multiple pieces (such as three or more pieces). Other types of fracture include complete, incomplete, linear, transverse, oblique, compression, spiral, comminuted, and compacted fractures. Additional fractures include a critical defect (such as when part of a bone is lost or removed) and a non-union fracture (such as when the ends of the fracture are not in contact with each other).

Isolated: An “isolated” biological component (such as a nucleic acid or protein or organelle) has been substantially separated or purified away from other biological components in the cell of the organism in which the component naturally occurs, e.g., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids or polypeptides.

Hybridization: Oligonucleotides and their analogs hybridize by hydrogen bonding, which includes Watson-Crick, Hoogsteen or reversed Hoogsteen hydrogen bonding, between complementary bases. Generally, nucleic acid consists of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C. “Complementary” refers to the base pairing that occurs between to distinct nucleic acid sequences or two distinct regions of the same nucleic acid sequence.

“Specifically hybridizable” and “specifically complementary” are terms that indicate a sufficient degree of complementarity such that stable and specific binding occurs between the oligonucleotide (or its analog) and the DNA or RNA target. The oligonucleotide or oligonucleotide analog need not be 100% complementary to its target sequence to be specifically hybridizable. An oligonucleotide or analog is specifically hybridizable when binding of the oligonucleotide or analog to the target DNA or RNA molecule interferes with the normal function of the target DNA or RNA, and there is a sufficient degree of complementarity to avoid non-specific binding of the oligonucleotide or analog to non-target sequences under conditions where specific binding is desired, for example under physiological conditions in the case of in vivo assays or systems. Such binding is referred to as specific hybridization.

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ concentration) of the hybridization buffer will determine the stringency of hybridization, though waste times also influence stringency. Calculations regarding hybridization conditions required for attaining particular degrees of stringency are discussed by Sambrook et al. (ed.), Molecular Cloning: A Laboratory Manual, 2nd ed., vol. 1-3, Cold Spring Harbor Laboratory Press, Cold Spring Harbor, N.Y., 1989, chapters 9 and 11, herein incorporated by reference.

Osteoblast: A mononucleate cell that is responsible for bone formation. Osteoblasts produce osteoid, which is composed mainly of Type I collagen. Osteoblasts are also responsible for mineralization of the osteoid matrix. Bone is a dynamic tissue that is constantly being reshaped by osteoblasts, which build bone, and osteoclasts, which resorb bone. Osteoblasts arise from osteoprogenitor cells located in the periosteum and the bone marrow. Osteoprogenitors are immature progenitor cells that express the master regulatory transcription factor Cbfa1/Runx2. Once osteoprogenitors start to differentiate into osteoblasts, they begin to express a range of markers including osterix, collagen type 1, alkaline phosphatase, osteocalcin, osteopontin, and osteonectin.

Osteoclast: A type of bone cell that removes bone tissue by removing its mineralized matrix by a process of bone resorption. Osteoclasts are formed by the fusion of cells of the monocyte-macrophage cell line. Osteoclasts are characterized by high expression of tartrate resistant acid phosphatase and cathepsin K.

Osteocyte: Mature, non-dividing bone cells that are housed in their own lacunae (small cavities in the bone). Osteocytes are derived from osteoblasts and they represent the final stage of maturation of the bone cell lineage. They are less active than osteoblasts, and although they are not responsible for a net increase in bone matrix, they are essential to the maintenance and routine turnover of the matrix. The narrow, cytoplasmic processes of osteocytes remain attached to each other and to osteoblasts through canaliculi (small channels in the bone).

Polypeptide: A polymer in which the monomers are amino acid residues which are joined together through amide bonds. When the amino acids are alpha-amino acids, either the L-optical isomer or the D-optical isomer can be used. The terms “polypeptide” or “protein” as used herein are intended to encompass any amino acid sequence and include modified sequences such as glycoproteins. The term “polypeptide” is specifically intended to cover naturally occurring proteins, as well as those which are recombinantly or synthetically produced.

Purified: The term purified does not require absolute purity; rather, it is intended as a relative term. Thus, for example, a purified peptide or polynucleotide preparation is one in which the peptide or polynucleotide is more enriched than the peptide or polynucleotide is in its natural environment within a cell. In one embodiment, a preparation is purified such that the protein or polynucleotide represents at least 50% of the total peptide or polynucleotide content of the preparation.

Repair: New bone formation which is sufficient to at least partially fill a void or structural discontinuity at the site of a bone defect. The term repair does not require a process of complete healing or a treatment which is 100% effective at restoring a defect to its pre-defect state.

RNA (ribonucleic acid): RNA is a long chain polymer which consists of nucleic acids joined by 3′-5′ phosphodiester bonds. The repeating units in RNA polymers are four different nucleotides, each of which comprises one of the four bases, adenine, guanine, cytosine, and uracil bound to a ribose sugar to which a phosphate group is attached. In general, DNA is transcribed to RNA by an RNA polymerase. RNA transcribed from a particular gene contains both introns and exons of the corresponding gene; this RNA is also referred to as pre-mRNA. RNA splicing subsequently removes the intron sequences and generates a messenger RNA (mRNA) molecule, which can be translated into a polypeptide. Triplets of nucleotides (referred to as codons) in an mRNA molecule code for each amino acid in a polypeptide, or for a stop signal.

Stem cell: A cell that can generate a fully differentiated functional cell of more than one given cell type. The role of stem cells in vivo is to replace cells that are destroyed during the normal life of an animal. Generally, stem cells (for example, embryonic stem cells) can divide without limit and are totipotent. After division, the stem cell may remain as a stem cell, become a precursor cell, or proceed to terminal differentiation. A multipotent stem cell is a stem cell that can generate a fully differentiated cell of more than one given cell type, but is not totipotent. In one example, a multipotent stem cell includes a mesenchymal cell that can self-renew and can generate bone-forming or mineral-forming cells, such as osteoblasts. Multipotent stem cells may be derived from tissues of a post-natal subject, for example, from bone marrow and adipose tissue; examples of multipotent stem cells include mesenchymal stem cells and adipose-derived stem cells.

An osteogenic cell is a cell that can generate a fully differentiated functional bone cell of at least one given cell type from the body of an animal, such as a human. An osteogenic cell can generate a fully differentiated bone cell, such as, but not limited to, an osteocyte, pre-osteoblast, osteoblast, pre-osteoclast, and/or osteoclast. After division, an osteogenic cell can remain a precursor cell (e.g. a cell that can generate a fully differentiated functional cell of at least one given cell type from the body of an animal), or may proceed to terminal differentiation. An osteogenic cell can give rise to one or more types of bone cells, such as osteocytes, pre-osteoblasts, osteoblasts, pre-osteoclasts, and/or osteoclasts, but is more limited in its ability to differentiate than a stem cell.

Subject: Living multi-cellular vertebrate organisms, a category that includes both human and veterinary subjects, including human and non-human mammals.

Substrate: A substance, framework, scaffold, or support on which cells may be grown and/or differentiated. The cells may be attached to the exterior of the substrate or may migrate into the substrate, for example into pores in the substrate. In some examples, a substrate is a sponge, strip, gel (such as a hydrogel), scaffold, or other three-dimensional structure. In a particular example, the substrate is a collagen sponge or a nylon strip.

Three-dimensional culture system: A system or apparatus for cell culture that allows contact of all sides of a cell or a substrate seeded with cells with the culture medium. In one example, a three-dimensional culture system is a rotating wall vessel (RWV), which is a rotating bioreactor for cell culture which is optimized to produce laminar flow and minimize mechanical stress on cells in culture. In the RWV system, the force of gravity is counterbalanced by mechanical forces, thereby simulating microgravity conditions. In another example, a three-dimensional culture system is a random positioning machine (also referred to as a 3-D clinostat), an instrument that randomly changes the position of a sample in three-dimensional space.

Treatment: Refers to a therapeutic intervention that ameliorates a sign or symptom of a disease or pathological condition. “Ameliorating” refers to the reduction in the number or severity of signs or symptoms of a disease or condition. In one embodiment, the disease or condition is a bone defect, such as a fracture, for example, a critical defect or non-union fracture.

### II. Overview of Several Embodiments

Disclosed herein are methods of making a biocompatible bone repair system which includes culturing multipotent stem cells with a first medium including a fibroblast growth factor (such as fibroblast growth factor-2) in the substantial absence of dexamethasone; seeding a biocompatible substrate including extracellular matrix (such as at least one ECM component, for example, fibrinogen) with the cultured multipotent stem cells; and inducing the multipotent stem cells seeded on the biocompatible substrate to differentiate into osteogenic cells by culturing the substrate seeded with the cultured multipotent stem cells with a second medium including a differentiation factor (such as a bone morphogenetic protein, for example, bone morphogenetic protein-2) in the substantial absence of dexamethasone.

Also provided are methods of treating a bone defect including culturing multipotent stem cells with a first medium including a fibroblast growth factor (such as fibroblast growth factor-2) in the substantial absence of dexamethasone; seeding a biocompatible substrate comprising extracellular matrix (such as at least one ECM component, for example, fibrinogen) with the cultured multipotent stem cells; inducing the multipotent stem cells seeded on the biocompatible substrate to differentiate into osteogenic cells by culturing the substrate seeded with the cultured multipotent stem cells with a second medium including a differentiation factor (such as a bone morphogenetic protein, for example, bone morphogenetic protein-2) in the substantial absence of dexamethasone; and surgically implanting the biocompatible substrate with the differentiated osteogenic cells at the bone defect site.

In the disclosed methods, multipotent stem cells are cultured in a first medium which contains a fibroblast growth factor (FGF) in the substantial absence of dexamethasone. In some examples, the FGF is any one of FGF-1 through FGF-23, or a combination thereof (such as FGF-2 or FGF-9). In a particular example, the fibroblast growth factor is FGF-2.

The FGF is included in the medium at an amount sufficient to stimulate expansion of the cell population. In some examples, FGF-2 is included in the medium at a concentration of at least about 0.1 ng/ml to about 20 ng/ml, such as about 0.1 ng/ml to about 10 ng/ml, about 0.2 ng/ml to about 8 ng/ml, about 1 ng/ml to about 10 ng/ml, about 2 ng/ml to about 5 ng/ml, or about 5 ng/ml. In one example, the concentration of FGF-2 in the medium is about 2.5 ng/ml. In some examples, the FGF is recombinant protein (such as human or mouse recombinant FGF). In a particular example, the FGF is human recombinant FGF-2. The medium may also contain additional components that promote or support survival or growth of the cells, such as fetal bovine serum (FBS), L-glutamine, buffers, glucose, or other components well known to one of skill in the art.

Multipotent stem cells are cultured in a medium containing an FGF (such as FGF-2) in the substantial absence of dexamethasone for a period of time sufficient for the cells to reach confluency or to reach a density or cell number that is sufficient to populate a biocompatible substrate, such as about 1×10³ to about 1×10⁹ cells, such as about 1×10⁵ to about 1×10⁷ cells, for example, about 2×10⁶ cells. In some examples, the cell number is determined directly, for example by counting the cells (such as with a hemocytometer) or by an assay that quantitates cell number (for example, CYQUANT™ Cell Proliferation assay; Invitrogen, Carlsbad, Calif.). In other examples, cell number can be determined indirectly, such as by DNA synthesis (for example by incorporation of [³H]thymidine) or by total protein content of a cell culture (for example using a Bradford protein assay, such as Bio-Rad DC™ kit; Bio-Rad, Hercules, Calif.).

In other examples, the cells are cultured in medium containing a FGF (such as FGF-2) in the substantial absence of dexamethasone for a pre-determined period of time, such as about 1 day to about 20 days. In particular examples, multipotent stem cells are cultured in a medium containing FGF-2 for about 1 day to about 20 days, such as about 3 days to about 15 days, about 5 days to about 10 days, or about 7 days.

The first cell culture medium contains an FGF, such as FGF-2, in the substantial absence of dexamethasone. In examples provided herein, cell culture medium utilized for the growth of multipotent stem cells includes FGF-2 in the substantial absence of dexamethasone (such as medium which does not contain added or exogenous dexamethasone, medium that contains less than about 10 nM dexamethasone (such as less than about 5 nM, less than about 1 nM, or less than about 0.1 nM dexamethasone), or medium that does not contain detectable levels of dexamethasone). Methods to detect dexamethasone are known in the art and include immunoassay (for example ELISA) and mass spectrometry.

In some examples, culture of the multipotent stem cells with the first medium is carried out using standard cell culture techniques, such as in a dish or multi-well plate. In other examples, the cells are cultured in suspension (such as in a spinner flask or rotating vessel) or under conditions that simulate microgravity (such as a rotating wall vessel or random positioning machine; see, e.g., Hammond and Hammond, Am. J. Physiol. 281:F12-F25, 2001; U.S. Pat. No. 7,291,500). The culture conditions are those appropriate for the cells in culture, for example, culture at about 37° C. in a humidified atmosphere of about 5% CO₂.

In some examples, changes in gene expression may be assessed during or following culture of the multipotent stem cells with medium containing an FGF in the substantial absence of dexamethasone. In some examples, culture of multipotent stem cells with an FGF in the substantial absence of dexamethasone increases expression of genes (such as proliferation genes, for example Cox-2, FGF-2, TGFβ, MMP3, VEGFA, VEGFR1, and EGR-1) as compared to multipotent stem cells which have not been cultured in medium with an FGF in the substantial absence of dexamethasone (such as an increase of about 2-fold to about 50-fold, for example, about 2-fold, about 5-fold, about 10-fold, about 15-fold, about 20-fold, about 30-fold, about 40-fold, about 50-fold). In other examples, culture of multipotent stem cells with an FGF in the substantial absence of dexamethasone decreases expression of genes (such as mineralization genes, for example BMP-2, OC, Noggin, RUNX2, Col1A1, and IGF-1) as compared to multipotent stem cells which have not been cultured in medium with an FGF in the substantial absence of dexamethasone (such as a decrease of about 10%, about 20%, about 30%, about 40%, about 50%, about 60% about 70%, about 80%, about 90%, or even about 95%).

Following culture of multipotent stem cells in the first medium as described above, the cells are seeded on a biocompatible substrate which includes ECM (such as at least one ECM component, for example, fibrinogen). Seeding the substrate includes introducing cells to the substrate such that they are in contact with or attached to the substrate. Suitable substrates include for example, a sponge, a strip, a scaffold, a gel, or a three-dimensional implant and may include collagen or gelatin, hyaluronic acid, polymers (such as nylon or polyester), polymer-hyaluronic acid, polymer-bioactive glass, tricalcium phosphate, hydroxyapatite surfaces, or other biologically compatible components or other scaffolds. Methods of seeding the substrate include incubating the substrate with a cell suspension, placing a cell suspension in contact with the substrate and allowing the cells to soak into the substrate, or placing the substrate in contact with a cell suspension and applying a vacuum.

In some examples, a substrate is incubated in a cell suspension (such as cells suspended in a culture medium) for a period of time sufficient for the cells to penetrate or attach to the substrate. The cell suspension may contain about 1×10³ to about 1×10⁹ cells, such as about 1×10⁵ to about 1×10⁷ cells, for example, about 2×10⁶ cells. In some examples, the substrate is incubated in a cell suspension for about one day to about ten days, such as about three days to about seven days, for example, about five days or about seven days.

In additional examples, a cell suspension is applied directly to a surface of a substrate and the suspension is allowed to soak into the substrate (for example a sponge). In some examples a cell suspension of about 100,000 cells per mm substrate to about 5 million cells per mm substrate, such as about 500,000 cells per mm substrate to about 3 million cells per mm substrate, or about 1 million cells per mm substrate to about 2 million cells per mm substrate is applied to a surface of a substrate.

In further examples, the substrate is contacted with cells (such as a cell suspension) and a vacuum is applied. In some examples, a vacuum of about 400 mm Hg is applied to draw the cells into the substrate. In other examples, the cells may be drawn into the substrate by gravity. In still further examples molecules that stimulate cell migration, such as an FGF (for example FGF-2) and/or ECM (for example fibronectin or fibrinogen) may be present on or in the substrate.

If the substrate is a non-porous material (such as a non-porous polymer, tricalcium phosphate, hydroxyapatite, or metal), the cells may be plated directly on the surface of the substrate and allowed to attach to the substrate surface.

The seeding of a substrate with cells can be assessed by methods known in the art. In some examples, the substrate is inspected visually for the presence of cells, for example by light microscopy (such as in the case of a transparent substrate). In other examples, the cells can be detected by immunofluorescence (such as by staining for the presence of cell proteins, for example actin). The cells may be attached to the outer surface of the substrate, or in the case of a porous substrate (such as a sponge or hydrogel), the cells may be present in the internal portion of the substrate, such as in the interstitial spaces of the substrate. In other examples, seeding of a substrate with cells can be assessed by staining cells using histological stains (such as hematoxylin and eosin), nucleic acid stains (such as nuclear staining, for example with 4′,6-diamidino-2-phenylindole (DAPI) or Hoechst stain), or by immunohistochemistry (for example, by staining for proteins expressed by the cells, such as actin).

In some examples, following seeding of multipotent stem cells on a biocompatible substrate including ECM, the substrate and cells are cultured for an additional period of time, such that the number of cells on the substrate increases, for example, until the cells reach confluency. In other examples, the substrate and cells are cultured for a pre-determined period of time, such as about 1 day to about 20 days, such as about 3 days to about 15 days, about 5 days to about 10 days, or about 7 days. In some examples, the substrate containing the cells is cultured in medium containing an FGF, such as FGF-2, in the substantial absence of dexamethasone. In other examples, the substrate containing the cells is cultured in a standard cell culture medium that promotes cell growth, such as αMEM.

In some examples, culture of the multipotent stem cells following seeding on a biocompatible substrate is carried out using standard cell culture techniques, such as in a cell culture flask, dish, or multi-well plate. In other examples, the substrate and cells are cultured under conditions that simulate microgravity (such as a rotating wall vessel or random positioning machine; see, e.g., Hammond and Hammond, Am. J. Physiol. 281:F12-F25, 2001; U.S. Pat. No. 7,291,500), such that all sides of the substrate and cells are exposed to the culture medium. The culture conditions are those appropriate for the particular cells in culture, for example, culture at about 37° C. in a humidified atmosphere of about 5% CO₂.

In other examples, following seeding of multipotent stem cells on the substrate, the substrate and cells are immediately cultured in a second medium containing a differentiation factor, such as a BMP, in the substantial absence of dexamethasone. In a particular example, the BMP is BMP-2.

In the disclosed methods, the cultured multipotent stem cells seeded on a biocompatible substrate with ECM are induced to differentiate to osteogenic cells by culturing in a second medium which contains a differentiation factor, such as a bone morphogenetic protein (BMP) in the substantial absence of dexamethasone. In some examples, the BMP may be any one of BMP-1 to BMP-15 (such as BMP-2, BMP-4 or BMP-7), or a combination of two or more thereof. In a particular example, the BMP is BMP-2.

The differentiation factor is included in the medium at an amount sufficient to induce differentiation of the multipotent stem cells to osteogenic cells. In some examples, BMP-2 is included in the medium at a concentration of at least about 10 ng/ml to about 200 ng/ml, such as about 20 ng/ml to about 200 ng/ml, about 50 ng/ml to about 100 ng/ml, or about 100 ng/ml. In some examples, the differentiation factor is a recombinant protein (such as human or mouse recombinant BMP). In a particular example, the BMP is human recombinant BMP-2.

The second cell culture medium contains a differentiation factor, such as BMP-2, in the substantial absence of dexamethasone. In examples provided herein, cell culture medium utilized for the differentiation of multipotent stem cells includes BMP-2 in the substantial absence of dexamethasone (such as medium which does not contain added or exogenous dexamethasone, medium that contains less than about 10 nM dexamethasone (such as less than about 5 nM, less than about 1 nM, or less than about 0.1 nM dexamethasone), or medium that does not contain detectable levels of dexamethasone). Methods of detecting dexamethasone are well known in the art, such as immunoassay (for example ELISA) and mass spectrometry.

The medium containing a bone morphogenetic protein (such as BMP-2) may also contain additional components that promote differentiation of multipotent stem cells to osteogenic cells. These components include one or more of β-glycerophosphate, ascorbic acid, vitamin D3, high phosphate, IGF-1, IGF-2, retinoic acid, and statins, but is in the substantial absence of dexamethasone. The medium may also contain additional components that promote or support survival or growth of the cells, such as fetal bovine serum (FBS), L-glutamine, buffers, glucose, or other components well known to one of skill in the art.

In another example, the second medium used to induce differentiation of the multipotent stem cells to osteogenic cells may contain a differentiation factor other than a BMP. For example, instead of a BMP, the second medium may include an insulin-like growth factor (such as IGF-1 and/or IGF-2) at a concentration of about 1 nM to about 1 μM, such as about 2 nM to about 800 nM. The medium containing a differentiation growth factor may also contain additional components that promote differentiation of multipotent stem cells to osteogenic cells. These components include one or more of β-glycerophosphate, ascorbic acid, vitamin D3, high phosphate, retinoic acid and statins.

The multipotent stem cells seeded on a biocompatible substrate including ECM are cultured in a medium containing a differentiation factor (such as BMP-2) in the substantial absence of dexamethasone for a period of time sufficient to induce the multipotent stem cells to differentiate to osteogenic cells. Methods of determining differentiation of stem cells to osteogenic cells are well known in the art (see e.g. Qi et al., Proc. Natl. Acad. Sci. USA 100:3305-10, 2003; de Jong et al., J Bone Miner Res. 17:2119-29, 2002). In some examples, differentiation of cells is assessed by evaluating expression of genes involved in mineralization, such as osteocalcin, osteopontin, osteonectin, alkaline phosphatase, RUNX2, collagen type I, and fibronectin. Methods of assessing gene expression are well known in the art and include Northern blot, in situ hybridization, reverse-transcriptase PCR (RT-PCR), and quantitative real-time RT-PCR (qRT-PCR). An increase in mineralization-related gene expression (such as an increase of about 50% to about 10-fold, for example, an increase of about 2-fold to about 5-fold) as compared to non-differentiated cells (for example, cells that have not been cultured with a differentiation factor, such as freshly isolated multipotent stem cells or cells that have been cultured with an FGF (such as FGF-2) in the substantial absence of dexamethasone, but have not been cultured with a BMP (such as BMP-2)) indicates differentiation of the cells to osteogenic cells. In particular non-limiting examples, an increase in expression of collagen type I, fibronectin, alkaline phosphatase, and/or osteocalcin indicates cell differentiation.

In further examples, the differentiation of multipotent stem cells to osteogenic cells is determined by assessing the amount of mineralization related proteins expressed by the cells, such as collagen type I, alkaline phosphatase, and osteocalcin. Methods of determining protein expression levels are well known in the art, and include Western blotting and immunohistochemistry. An increase in mineralization-related protein expression (such as an increase of about 50% to about 10-fold, for example, an increase of about 2-fold to about 5-fold) as compared to non-differentiated cells (for example, cells that have not been cultured with a differentiation factor, such as freshly isolated multipotent stem cells or cells that have been cultured with an FGF (such as FGF-2) in the substantial absence of dexamethasone, but have not been cultured with a BMP (such as BMP-2)) indicates differentiation of the cells to osteogenic cells.

In additional examples, differentiation of the cells is determined by histological staining of calcium deposit in the culture, such as by staining with Alizarin Red S or Von Kossa stain. An increase in the presence of calcium by at least about 10%, 25%, 50%, 75%, 2-fold, 3-fold, 4-fold, 5-fold, 10-fold, 20-fold, 50-fold, or more as compared to non-differentiated cells (for example, cells that have not been cultured with a differentiation factor, such as freshly isolated multipotent stem cells or cells that have been cultured with an FGF (such as FGF-2) in the substantial absence of dexamethasone, but have not been cultured with a BMP (such as BMP-2)) indicates that the cells are differentiated to osteogenic cells.

Differentiation of cells may also be evaluated by assessing activity of proteins associated with mineralization. In one example, alkaline phosphatase activity is assessed, for example by staining with α-naphthyl phosphate or naphthyl AS phosphate and fast blue RR or fast violet B (e.g. leukocyte alkaline phosphatase kit, Sigma-Aldrich, St. Louis, Mo.). Alkaline phosphatase activity is determined semi-quantitatively by this method. See, e.g., Kaplow, Blood 10: 1023, 1955; Kaplow, Ann NY Acad Sci 155:911, 1968; Ackerman, Lab Invest 11:563, 1962. An increase in alkaline phosphatase activity (such as an increase of at least about 25%, about 50%, about 100%, about 5-fold, about 10-fold, or more) as compared to non-differentiated cells (for example, cells that have not been cultured with a differentiation factor, such as freshly isolated multipotent stem cells or cells that have been cultured with an FGF (such as FGF-2) in the substantial absence of dexamethasone, but have not been cultured with a BMP (such as BMP-2)) indicates that the cells are differentiated to osteogenic cells.

In some examples, the cells seeded on a biocompatible substrate including ECM are cultured in medium containing a differentiation factor (such as BMP-2) in the substantial absence of dexamethasone for a pre-determined period of time. In particular examples, the cells on the substrate are cultured in a medium containing BMP-2 in the substantial absence of dexamethasone for about 1 day to about 20 days, such as about 3 days to about 15 days, about 5 days to about 10 days, or about 7 days.

In some examples, culture of the multipotent stem cells seeded on a biocompatible substrate including ECM with the second medium is carried out using standard cell culture techniques, such as in a dish or multi-well plate. In other examples, the cells are cultured under conditions that simulate microgravity (such as a rotating wall vessel or random positioning machine), such that all sides of the substrate and cells are exposed to the culture medium. The culture conditions are those appropriate for the cells in culture, for example, culture at 37° C. in a humidified atmosphere of 5% CO₂.

### III. Extracellular Matrix

A substantial portion of the volume of tissues consists of extracellular space, which is filled with macromolecules that make up the extracellular matrix (ECM). The ECM is composed of a complex mixture of proteins, polysaccharides, and other components that are secreted by cells and are in close association with the cells. Functions of the ECM include providing structural support for cells and tissues and regulating cell behavior (such as migration, proliferation, development, and function). The major components of the ECM are polysaccharides (such as glycosaminoglycan chains), which are usually linked to proteins to form proteoglycans, and proteins, which are generally either structural or adhesive.

One component of the ECM is polysaccharide chains, such as glycosaminoglycan (GAG) chains. GAGs are unbranched polysaccharide chains made up of repeating disaccharide units The GAGs found in animals include hyaluronic acid (HA; also known as hyaluronan), chondroitin sulfate, dermatan sulfate, heparan sulfate, and keratan sulfate. HA is made up of repeating non-sulfated disaccharide units consisting of D-glucuronic acid and N-acetyl-D-glucosamine. The repeating disaccharide unit of chondroitin sulfate is D-glucuronic acid and N-acetyl-D-galactosamine; the repeating unit of heparan sulfate is D-glucosamine and N-acetyl-D-glucosamine; and the repeating unit of keratin sulfate is D-galactose and N-acetyl-D-glucosamine.

HA exists as long chains of up to 25,000 nonsulfated disaccharide subunits and is not linked to proteins. In contrast, the other GAGs are covalently attached to proteins as proteoglycans. Examples of proteoglycans and their GAG components include aggrecan (chondroitin sulfate and keratan sulfate), betaglycan (chondroitin sulfate and dermatan sulfate), decorin (chondroitin sulfate and dermatan sulfate), perlecan (heparan sulfate), serglycin (chondroitin sulfate and dermatan sulfate), and synecan-1 (chondroitin sulfate and heparan sulfate).

A major portion of the ECM consists of various proteins. ECM proteins include, but are not limited to, collagen (such as collagen Type I or collagen Type IV), elastin, fibrin, fibrillin, fibronectin, fibrinogen, laminin, entactin, tenascin, and agrin. The collagen family includes at least 15 types of collagen, which are major components of the ECM. Type I collagen is the principal collagen found in skin and bone and consists of two type I α1 collagen subunits and one type I α2 collagen subunit. Type IV collagen is a major component of the basal lamina and consists of two type IV α1 subunits and one type IV α2 subunit.

Disclosed herein are methods of making a biocompatible transplantable bone repair system which includes seeding osteogenic stem cells that have been cultured in medium containing FGF-2 in the substantial absence of dexamethasone on a biologically compatible substrate that includes ECM. In some examples, the ECM includes one or more ECM component, including, but not limited to collagens (such as collagen type I (COL1A1) and collagen type IV), elastin, entactin (EN), fibrin, fibrinogen, fibronectin (FN), fibrillin, agrin, chrondroitin sulfate, dermatan sulfate, keratin sulfate, heparin sulfate, hyaluronic acid (HA), laminin, and combinations of two or more thereof. In some examples, the ECM includes hyaluronic acid (such as HA and heparin (HAHp)). In particular examples, the ECM is fibrinogen.

In some examples, the biologically compatible substrate that includes ECM includes an amount of one or more ECM component that promotes migration or adhesion of the cells to the substrate or that results in an increased number of cells associated with the substrate. In particular examples, the ECM includes fibrinogen (such as about 10 ng/ml to about 10 mg/ml, for example, about 0.1 mg/ml, 0.2 mg/ml, 0.5 mg/ml, 1 mg/ml, 2 mg/ml, or 5 mg/ml). In other examples, the ECM includes HA, for example, about 0.1 mg/ml to about 10 mg/ml HA (such as about 0.5 mg/ml, 1 mg/ml, 2 mg/ml, or 5 mg/ml HA). In some examples, heparin is also included in the ECM (such as about 1 mg/ml to about 10 mg/ml, for example, about 2 mg/ml, 3 mg/ml, 5 mg/ml, or 10 mg/ml heparin). In a particular example, the ECM includes 1 mg/ml HA and 3 mg/ml heparin.

In additional examples, the ECM includes a mixture of at least two or more ECM components, such as at least two, at least three, at least four, or at least five ECM components. Some exemplary ECM mixtures are provided in Table 1. In a particular example, the ECM includes laminin, collagen type IV, and elastin (e.g. MATRIGEL™ (BD Biosciences, San Jose, Calif.)). In some examples, the ECM includes one or more proteoglycan (such as aggrecan, betaglycan, decorin, perlecan, serglycin, and synecan-1), or a combination of one or more proteoglycan and one or more ECM protein. In other examples, the ECM includes collagen and fibrinogen, gelatin and fibrinogen, collagen and HAHp, or gelatin and HAHp.

TABLE 1
Exemplary ECM Mixtures

| | COL1A1 | FN | HA | EN | Total mix |
|---|---|---|---|---|---|
| FN | 75% COL1A1 | 50% COL1A1 | 50% COL1A1 | 50% COL1A1 | 50% COL1A1 |
| | 25% FN | 50% FN | 25% FN | 25% FN | 16.66% FN |
| | | | 25% HA | 25% HA | 16.66% HA |
| | | | | | 16.66% EN |
| HA | 75% COL1A1 | 50% COL1A1 | 50% COL1A1 | 50% COL1A1 | |
| | 25% HA | 25% FN | 50% HA | 25% EN | |
| | | 25% HA | | 25% HA | |
| EN | 75% COL1A1 | 50% COL1A1 | 50% COL1A1 | 50% COL1A1 | |
| | 25% EN | 25% FN | 25% HA | 50% EN | |
| | | 25% EN | 25% EN | | |

COL1A1, type 1 collagen;
FN, fibronectin;
HA, hyaluronic acid;
EN, entactin

### IV. Biocompatible Substrates

The methods described herein include the use of a biocompatible substrate which is a component of the biocompatible transplantable bone repair system. The substrate is seeded with multipotent stem cells which have been cultured in a medium containing a FGF (such as FGF-2) in the substantial absence of dexamethasone. The stem cells are then induced to differentiate on the substrate by culturing the substrate seeded with the cells in a medium containing a differentiation factor (such as BMP-2) in the substantial absence of dexamethasone. In some examples, the biologically compatible substrate is surgically implanted in a subject at the site of a bone defect in order to treat the bone defect. All biocompatible substrates described herein include at least one ECM component (for example, fibrinogen).

In some examples, the biologically compatible substrate that includes ECM further includes additional components, such as one or more growth factors (such as an FGF). In one example, the substrate further includes FGF-2 at about 1 ng/ml to about 500 ng/ml, such as about 10 ng/ml, 20 ng/ml, 50 ng/ml, 75 ng/ml, 100 ng/ml, 150 ng/ml, 200 ng/ml, 250 ng/ml, 300 ng/ml, 400 ng/ml, or 500 ng/ml. In particular examples, the substrate that includes ECM further includes 100 ng/ml FGF-2.

The biocompatible substrate may include one or more proteins (such as collagen, for example, collagen type I or gelatin), polymers (for example, nylon or polyester), polymer-hyaluronic acid, polymer-bioactive glass, tricalcium phosphate, hyaluronic acid surfaces, hydroxyapatite, or mixtures thereof. In some examples, the substrate may be a fabric, such as nylon, silk, or DACRON® polyester (e.g., Barros D'Sa et al., Ann. Surg. 192:645-657, 1980). In other examples, the substrate may include metal, such as titanium.

The biologically compatible substrate may be in the form of a scaffold or other supporting structure, such as a sponge, strip, gel (such as a hydrogel), scaffold, or other three-dimensional structure. In one example, the substrate is a sponge. In a particular example, the biologically compatible substrate is a collagen or gelatin sponge (such as GELFOAM® (Pfizer, New York, N.Y.) or SURGIFOAM® (Johnson & Johnson, New Brunswick, N.J.)). In another particular example, the biologically compatible substrate is a nylon strip.

The substrate is contacted with at least one ECM component (as well as any additional components, such as an FGF) prior to seeding the substrate with cells. In some examples, the substrate is soaked in a solution containing the at least one ECM component (such as fibronectin or HAHp), such that the ECM coats or adheres to the substrate. The substrate is soaked in the ECM solution for sufficient time for the ECM to coat the substrate, for example for about 1 minute to about 30 minutes, about 5 minutes to about 15 minutes, about 10 minutes, or about 5 minutes. The ECM is in a solution such that there is sufficient ECM to coat the entire substrate (for example, 10 ng/ml to about 10 mg/ml, for example, about 25 ng/ml, 50 ng/ml, 100 ng/ml, 200 ng/ml, 500 ng/ml, 1 mg/ml, 2 mg/ml, or 5 mg/ml). In a particular example, a substrate is soaked in a solution of about 50 ng/ml fibrinogen for about 5 minutes. In other examples, the substrate is pre-treated with about 100 ng/ml fibrinogen and about 50 ng/ml FGF-2.

The substrate is generally a three-dimensional shape, such as a cube, rectangular block, triangle, wedge, or other appropriate shape. In particular examples, the substrate is shaped to fit the particular bone defect which is to be repaired. The substrate may also be essentially two-dimensional, such as a strip. The biocompatible substrate is of a size and shape suitable for repair of a particular bone defect in a subject. For example, a three-dimensional substrate may have dimensions of about 1 mm³ to about 50 mm³, such as about 2 mm³ to about 25 mm³, such as about 5 mm³ to about 10 mm³. In some examples, the substrate has dimensions of about 0.5 inches by 0.25 inches to about 1 inch by 1 inch. However, one of skill in the art will recognize that the dimensions of the substrate are determined by the dimensions of the bone defect which is to be repaired. In additional examples, the transplantable bone repair system can be prepared in dimensions that are larger than the bone defect and cut to the appropriate size prior to implanting in the defect.

Biocompatible substrates are well known in the art (see, e.g. U.S. Pat. Nos. 5,700,289 and 6,541,024). Examples of a biocompatible substrate include biodegradable and biocompatible polymer scaffolds (see Jang et al., Expert Rev. Medical Devices 1:127-138, 2004). These scaffolds usually contain a mixture of one or more biodegradable polymers, for example and without limitation, saturated aliphatic polyesters, such as poly(lactic acid) (PLA), poly(glycolic acid), or poly(lactic-co-glycolide) (PLGA) copolymers, unsaturated linear polyesters, such as polypropylene fumarate (PPF), or microorganism produced aliphatic polyesters, such as polyhydroxyalkanoates (PHA) (see Rezwan et al., Biomaterials 27:3413-3431, 2006; Laurencin et al., Clin. Orthopaed. Rel. Res. 447:221-236). By varying the proportion of the various components, polymeric scaffolds of different mechanical properties are obtained. A commonly used scaffold contains a ratio of PLA to PGA of about 75:25, but this ratio may change depending upon the specific application. Other commonly used scaffolds include surface bioeroding polymers, such as poly(anhydrides), such as trimellitylimidoglycine (TMA-gly) or pyromellitylimidoalanine (PMA-ala), or poly(phosphazenes), such as high molecular weight poly(organophosphazenes) (P[PHOS]), and bioactive ceramics.

Another class of materials for making a substrate is hydroxyapatite, or a ceramic formed of tricalcium phosphate (TCP) or calcium phosphate (CaPO₄). Calcium hydroxyapatites occur naturally as geological deposits and in normal biological tissues, principally bone, cartilage, enamel, dentin, and cementum of vertebrates and in many sites of pathological calcifications such as blood vessels and skin. Synthetic calcium hydroxyapatite is formed in the laboratory either as pure Ca₁₀(PO₄)₆(OH)₂ or hydroxyapatite that is impure, containing other ions such as carbonate, fluoride, chloride for example, or crystals deficient in calcium or crystals in which calcium is partly or completely replaced by other ions such as barium, strontium and lead.

Calcium phosphate ceramics can be used as implants in the repair of bone defects because these materials are non-toxic, non-immunogenic, and are composed of calcium and phosphate ions, the main constituents of bone. Both tricalcium phosphate (TCP) [Ca₃(PO₄)₂] and hydroxyapatite [Ca₁₀(PO₄)₆(OH)₂] have been widely used. Calcium phosphate implants are osteoconductive, and have the apparent ability to become directly bonded to bone.

In other examples, polymers that can form ionic hydrogels which are malleable can also be used as a biocompatible substrate. A hydrogel may be utilized to deliver cells and promote the formation of new tissue without the use of any other substrate. In one example, the hydrogel is produced by cross-linking the ionic salt of a polymer with ions, whose strength increases with either increasing concentrations of ions or polymer.

A hydrogel is defined as a substance formed when an organic polymer (natural or synthetic) is cross-linked via covalent, ionic, or hydrogen bonds to create a three-dimensional open-lattice structure which entraps water molecules to form a gel. Examples of materials which can be used to form a hydrogel include polysaccharides such as alginate, polyphosphazenes, and polyacrylates such as hydroxyethyl methacrylate, which are crosslinked ionically, or block copolymers such as PLURONICS™ (BASF Corporation) or TETRONICS™ (BASF Corporation), polyethylene oxide-polypropylene glycol block copolymers which are crosslinked by temperature or pH, respectively. Other materials include proteins such as fibrin, polymers such as polyvinylpyrrolidone, hyaluronic acid and collagen.

The biologically compatible substrate may also include gelatin, cellulose, or collagen-based materials. In some examples, the gelatin-based substrate includes an absorbable sponge, powder or film of cross-linked gelatin, for example, GELFOAM® (Upjohn, Inc., Kalamazoo, Mich.) which is formed from denatured collagen. A cellulose-based substrate includes an appropriate absorbable cellulose such as regenerated oxidized cellulose sheet material, for example, SURGICEL® (Johnson & Johnson, New Brunswick, N.J.) or Oxycel® (Becton Dickinson, Franklin Lakes, N.J.). A biologically compatible collagen-based substrate includes an appropriate resorbable collagen, such as purified bovine corium collagen, for example, AVITENE® (MedChem, Woburn, Mass.), HELISTAT® (Marion Merrell Dow, Kansas City, Mo.), HEMOTENE® (Astra, Westborough, Mass.), or SURGIFOAM® (Johnson & Johnson, New Brunswick, N.J.).

### V. Stem Cells

The methods disclosed herein utilize multipotent stem cells for the preparation of a biocompatible transplantable bone repair system. The multipotent stem cells are stem cells, such as adult stem cells, that have the capacity to generate a fully differentiated cell of more than one given cell type. However, multipotent stem cells do not have the capacity to generate all fully differentiated cell types of an animal; they are not totipotent, as are embryonic stem cells. Multipotent stem cells are present in low numbers in post-natal animals, such as immature, adolescent, or adult animals, such as mice or humans. These multipotent stem cells can be purified from a tissue and induced to differentiate into a variety of fully differentiated cell types, depending on the source of the multipotent stem cell and the conditions the stem cell is exposed to.

Mesenchymal stem cells (MSCs) can be obtained from bone marrow, for example, at sites such as the iliac crest, femora, tibiae, spine, rib or other medullary spaces. Other sources of MSCs include placenta, umbilical cord, periosteum, skin, and blood. MSCs, such as MSCs from bone marrow, have the potential to differentiate into multiple cell types, such as osteoblasts, adipocytes, and chondrocytes (see e.g. Phinney and Prockop, Stem Cells 25:2896-2902, 2007; Chamberlain et al., Stem Cells 25:2739-2749, 2007). In particular examples, MSCs are obtained from bone marrow. Autologous or allogenic MSCs may be used in the methods provided herein. In a preferred example, the MSCs are autologous to a subject in need of bone repair.

In some examples, the expression of the antigens CD34, CD59, CD45, CD90, and CD105 are used to identify a population of MSCs useful for the methods disclosed herein. An antigen expression profile can be generated from a donor by fluorescent-activated cell sorting analysis. In some examples, MSC cultures that are positive (such as greater than about 75%) for CD59, CD90 and CD105, and negative (such as less than about 1%) for CD45 and CD34 are used for the methods disclosed herein. In some examples MSC cultures from donors that have the ability to produce more than about 100 CFU-F per 10⁷ plated cells are used (Castro-Malaspina et al., Prog Clin Biol Res. 154:209-36, 1984).

Multipotent stem cells can also be obtained from adipose tissue (such as subcutaneous white adipose, for example adipose tissue from abdomen, breast, thigh, or arm). These cells are referred to as processed lipoaspirate cells (see Zuk et al., Mol. Biol. Cell 13:4279-4295, 2002) or adipose-derived stem cells (ASCs) (see Gimble et al., Circ. Res. 100:1249-1260, 2007). ASCs have the potential to differentiate into multiple cell types, including adipocytes, chondrocytes, osteoblasts, and neuronal-like cells. Methods of isolating ASCs are known to one of skill in the art, for example from a pelleted stromal vascular fraction prepared from lipoaspirate. See, e.g. Gimble et al., Circ. Res. 100:1249-1260, 2007. Autologous or allogenic ASCs may be used in the methods provided herein. In a preferred example, the ASCs are autologous to a subject in need of bone repair.

In some examples, the expression of the antigen CD34 is used to identify a population of ASCs useful for the methods disclosed herein. An antigen expression profile can be generated from a donor by fluorescent-activated cell sorting analysis. In some examples, ASC cultures that are positive (such as greater than about 75%) for CD34 are used for the methods disclosed herein. In some examples ASC cultures from donors that have the ability to produce more than about 100 CFU-F per 10⁷ plated cells are used (Castro-Malaspina et al., Prog. Clin. Biol. Res. 154:209-36, 1984).

### VI. Methods of Treating Bone Defects

Methods are provided to treat bone defects utilizing the biocompatible transplantable bone repair system described herein. The methods include preparing a biocompatible transplantable bone repair system as described above. The bone repair system is surgically implanted at the site of a bone defect in order to treat the bone defect. In some examples, the bone repair system is sized to fit the defect to be repaired. In particular examples, the bone repair system is cemented in place (such as with hyaluronic acid or superglue), for example in a bone defect in the skull. In other examples, the bone repair system is fastened in place, such as with metal or plastic screws. In some examples, the substrate includes fabric (such as nylon, silk, or DACRON®) which may be stretched across a fracture or placed inside a defect as multiple layers (for example, in a large fracture or a non-union fracture). The methods can be used in human or non-human subjects.

The bone defect can be a fracture, such as a critical defect or non-union fracture. A fracture is a condition in which a bone is cracked or broken; a break in the continuity of a bone. Fractures may be classified as closed (such as when the skin is intact) or open (exposed to the air, such as piercing the skin or due to severe tissue injury). Fractures are also classified as simple or multi-fragmentary. A simple fracture occurs along only one line (such as splitting a bone into two pieces), while a multi-fragmentary (or comminuted) fracture splits a bone into multiple pieces (such as three or more pieces). Other types of fracture include complete (such as when bone fragments are completely separated), incomplete (such as when bone fragments are at least partially in contact), linear (such as a fracture parallel to the long axis of a bone), transverse (such as a fracture at right angles to the long axis of a bone), oblique (such as a fracture diagonal to the long axis of a bone), compression (such as collapse of a vertebrae, for example as a result of osteoporosis), spiral (such as when at least one portion of the fractured bone is twisted), and compacted (such as a fracture resulting from bone fragments being driven into one another). In children, fractures also include greenstick fractures (such as a fracture in which the bone does not completely fracture, but exhibits bowing without complete disruption of the cortex of the bone). In particular examples, fractures include a critical defect (such as when part of a bone is lost or removed) and a non-union fracture (such as when the ends of the fracture are not in contact with each other).

The defect or fracture can be in any bone, including but not limited to cranial bones such as the frontal bone, parietal bone, temporal bone, occipital bone, sphenoid bone, ethmoid bone; facial bones such as the zygomatic bone, superior and inferior maxilla, nasal bone, mandible, palantine bone, lacrimal bone, vomer bone, the inferior nasal conchae; the bones of the ear, such as the malleus, incus, stapes; the hyoid bone; the bones of the shoulder, such as the clavicle or scapula; the bones of the thorax, such as the sternum or the ribs; the bones of the spinal column including the cervical vertebrae, lumbar vertebrae, and thoracic vertebrae; the bones of the arm, including the humerus, ulna and radius; the bones of the hands, including the scaphoid, lunate, triquetrum bone, psiform bone, trapezium bone, trapezoid bone, cpitate bone, and hamate bone; the bones of the palm such as the metacarpal bones; the bones of the fingers such as the proximal, intermediate and distal phalanges; the bones of the pelvis such as the ilium, sacrum and coccyx; the bones of the legs, such as the femur, tibia, patella, and fibula; the bones of the feet, such as the calcaneus, talus, navicular bone, medial cuneiform bone, intermediate cuneiform bone, lateral cuneiform bone, cuboidal bone, metatarsal bone, proximal phalanges, intermediate phalanges and the distal phalanges; and the pelvic bones. In one example, a bone fracture is repaired in the absence of extra-skeletal bone formation, such as in the absence of bone formation in the soft tissues.

Methods are also provided to treat bone defects by promoting spinal fusion. Spinal fusion can be induced in any of the vertebrae, including, but not limited to, the cervical vertebrae, lumbar vertebrae, and thoracic vertebrae. In one example, spinal fusion occurs in the absence of extra-skeletal bone formation, such as in the absence of bone formation in the soft tissues. In other examples, methods are provided to treat dental or facial bone defects, such as cleft palate, jaw injuries or defects, and facial fractures.

The natural process of healing a fracture starts when the injured bone and surrounding tissues bleed. The blood coagulates to form a blood clot situated between the broken fragments. Within a few days blood vessels grow into the jelly-like matrix of the blood clot. The new blood vessels bring white blood cells to the area, which gradually remove the non-viable material. The blood vessels also bring fibroblasts in the walls of the vessels and these multiply and produce collagen fibers. In this way the blood clot is replaced by a matrix of collagen.

At this stage, some of the fibroblasts begin to lay down bone matrix (calcium hydroxyapatite) in the form of insoluble crystals. This mineralization of the collagen matrix stiffens it and transforms it into bone. Healing bone callus is on average sufficiently mineralized to show up on X-ray within 6 weeks in adults and less in children. This initial “woven” bone does not have the strong mechanical properties of mature bone. By a process of remodeling, the woven bone is replaced by mature “lamellar” bone. The whole process can take up to 18 months, but in adults the strength of the healing bone is usually 80% of normal by 3 months after the injury. In some types of fracture (such as critical defect or non-union fracture), the normal process of bone healing do not occur or occur very slowly.

In some embodiments, the biocompatible transplantable bone repair device disclosed herein is used to treat subjects that have a bone defect due to any disease, defect, or disorder which affects bone strength, function, and/or integrity, such as decreasing bone tensile strength and modulus. Examples of bone diseases include, but are not limited to, diseases of bone fragility, such as osteoporosis. Other examples include bone defects in a subject affected with malignancies and/or cancers of the bone such as a sarcoma, such as osteosarcoma.

Assays to determine if a transplanted bone repair device treats the bone defect are known in the art. Non-limiting examples of suitable assays include radiographic methods (Lehmann et al., Bone 35: 1247-1255, 2004; Rundle et al., Bone 32: 591-601, 2003; Nakamura et al., J. Bone Miner. Res. 13: 942-949, 1998); microcomputed tomography (μCT) methods (Nakamura et al., J. Bone Miner. Res. 13: 942-949, 1998; Lehmann et al., Bone 35: 1247-1255, 2004; Tamasi et al., J. Bone Miner. Res. 18: 1605-1611, 2003; Shefelbine et al., Bone 36:480-488, 2005); peripheral quantitative computed tomographic methods (Rundle et al., Bone 32: 591-601, 2003; Tamasi et al., J. Bone Miner. Res. 18: 1605-1611, 2003); dual energy X-ray absorptiometry methods (Holzer et al., Clin. Orthop. Rel. Res. 366: 258-263, 1999; Nakamura et al., J. Bone Miner. Res. 13: 42-949, 1998); histomorphometry methods (Lehmann et al., Bone 35: 247-1255, 2004; Tamasi et al., J. Bone Miner. Res. 18:1605-1611, 2003; Li et al., J. Bone Miner. Res. 17: 791-799, 2002; Schmidmaier et al., Bone 30: 816-822; 2002; Nakamura et al., J. Bone Miner. Res. 13:942-949, 1998; Sheng et al., Bone 30: 486-491, 2002); Masson's trichrome stain for collagen (Rundle et al., Bone 32: 591-601, 2003); Goldner's stain for collagen (Holzer et al., Clin. Orthop. Rel. Res. 366: 258-263; 1999); Von Kossa's silver stain for bone (Schmidmaier et al., Bone 30: 816-822, 2002); Safranin Orange stain for collagen (Schmidmaier et al., Bone 30: 816-822, 2002); and immunohistochemistry methods (Rundle et al., Bone 32: 591-601, 2003; Li et al., J. Bone Miner. Res. 17: 791-799, 2002; Safadi et al., J. Cell Physiol. 196: 51-62, 2003; Iwaki et al., J. Bone Miner. Res. 12: 96-102, 1997).

Treating a bone defect includes stimulation of new bone formation which is sufficient to at least partially fill a void or structural discontinuity at the site of a bone defect. Treatment of the bone defect does not require a process of complete healing or a treatment which is 100% effective at restoring a defect to its pre-defect state. Successful treatment of a bone defect includes partial repair or healing, for example filling of at least 10%, at least 20%, at least 30%, at least 40%, at least 50%, at least 60%, at least 70%, at least 80%, at least 90%, at least 95%, or at least 99% of the bone defect with new bone material.

The present disclosure is illustrated by the following non-limiting Examples.

### EXAMPLES

### Example 1

### Effect of Growth Factors on Cell Growth and Migration in an In Vitro Fracture Model

This example describes the effects of growth factors on the growth and migration of MC3T3-E1 cells and human mesenchymal stem cells in an in vitro uniform bone fracture model.

### Methods

Cell culture: Cloned MC3T3-E1 osteoblasts (Kodama et al., J. Cell. Physiol. 112:89-95, 1992) were maintained in alpha MEM medium (Catalog No. MT15012CV Fisher Scientific, Pittsburgh, Pa.) containing 10% fetal bovine serum (FBS), 1% antibiotic solution and 1% glutamine solution and subcultured every 3 to 4 days. The cells were subcultured by incubating with trypsin for five minutes and resuspending at a concentration of 3×10⁵ cells/ml. For experiments, cells were grown in the same medium as above, using multiwell plates (35 mm for microscopy and 22 mm for thymidine uptake studies). Each well contained a round 12 mm glass cover slip. Cultures for microscopy contained a 22 mm diameter coverslip with a 12 mm coverslip on top. After three days, the cells reached confluence and differentiation medium (DM) was added. DM was alpha MEM medium containing 10% FBS, 1% antibiotic solution and 1% glutamine solution, supplemented with ascorbic acid (50 μg/ml) and β-glycerol phosphate (10 mM) to induce mineralization. The cultures were then incubated for 9 more days. Microscopic examination was done at this time, using Von Kossa stain to confirm mineralization of the cultures. A critical defect in the mineralized cell layers was created by removing the 12 mm coverslips under sterile conditions and then incubating the cultures for an additional 24 hours in DM containing 4% FBS with IGF-1 (20 ng/ml), FGF-2 (0.2 ng/ml), PDGF (3 ng/ml), TGFβ (2 ng/ml) or PGE₂ (2 μg/ml). At least triplicate independent biological samples in multiple experiments were carried out for data collection.

Mesenchymal stem cells were isolated from bone marrow by adding 50 μl ROSETTESEP® human mesenchymal stem cell enrichment cocktail (StemCell Technologies, Vancouver, BC, Canada) to each ml of bone marrow. The mixture was incubated for 20 minutes at room temperature and then diluted 2× with PBS containing 2% FBS and 1 mM EDTA. The diluted sample was layered on FICOLL-PAQUE™ (StemCell Technologies, Vancouver, BC, Canada) and centrifuged at 300×g for 25 minutes. Enriched MSCs were removed from the FICOLL-PAQUE™:plasma interface, washed with PBS containing 2% FBS and 1 mM EDTA, and resuspended in αMEM culture medium.

Isolated MSCs were seeded to 96-well plates. Once the cells adhered, they were down-regulated for 70 hr in αMEM medium with 3% FBS. 2,000 cells per well were seeded on 96-well plates. FGF-2 was added to the culture and cells were harvested 48 hr after the beginning of each treatment. Relative cell number was obtained with a CYQUANT® Cell Proliferation kit (Invitrogen, Carlsbad, Calif.) according to the manufacturer's protocol.

Microscopy: At the conclusion of the 24 hour incubation, the remaining 22 mm coverslip containing the regenerating area of the culture was removed. The specimen was rinsed five times in room temperature phosphate buffered saline (PBS) and fixed. Cells were visualized with the F-actin probe rhodamine phalloidin (Invitrogen, Carlsbad, Calif.). After rinsing in distilled water and air drying the samples, the coverslips were mounted on microscope slides using FLUOROMOUNT™ and the cells were examined and photographed. Photomicrographs were made on a Zeiss Axioskop epi-fluorescent microscope using a 40× objective. Migration was measured on the photomicrographs from the site of the wound to the migrating edge using a metric ruler (n=3) for each measurement.

Tritiated thymidine incorporation into DNA: At the conclusion of the 24 hour incubation, the culture medium was removed and the cells were incubated for 15 minutes at 37° C. in 1 ml PBS containing tritiated thymidine (4 μCi/ml). Following this incubation, the PBS was removed and the cells washed 3 times with ice cold trichloroacetic acid (TCA) followed by ice cold ethanol and allowed to air dry. Then 1 ml of sarkosyl lysis buffer (10 μM Tris-Cl (pH 7.5), 10 μM EDTA, 10 μM NaCl containing 1% sarkosyl) was added to each well; all the cells were solubilized after 30 minutes. Finally, after mixing the resulting solution with a pipette, radioactivity was counted in a scintillation counter and protein content was measured. The data was calculated and expressed as disintegrations per minute per microgram protein.

Protein assay: Protein concentration was determined by Bio-Rad DC™ protein assay (Bio-Rad, Hercules, Calif.) according to the manufacturer's protocol.

### Results

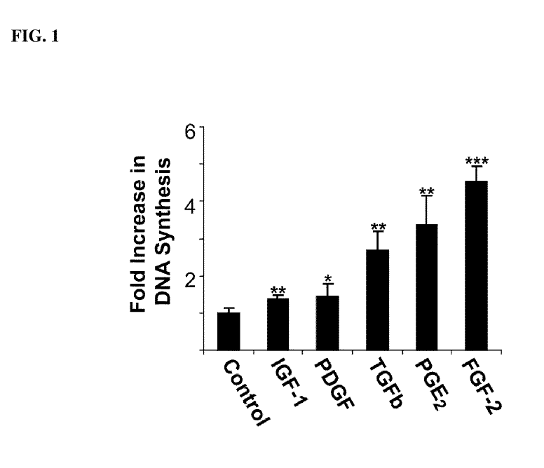

In the absence of any added compounds, wounding alone significantly increased DNA synthesis. There were small and unremarkable changes in DNA synthesis with IGF-1 and PDGF; in contrast, FGF-2, TGFβ, and PGE₂ significantly enhanced thymidine incorporation (FIG. 1) within twenty-four hours of treatment. TGFβ stimulated thymidine incorporation more than 2-fold while FGF-2 and PGE₂ increased thymidine incorporation more than 4.5- and 3.3-fold, respectively.

The relative percent increase in protein content of the wounded and control cultures are shown in Table 2; values are normalized to the control culture. Addition of the active growth factors increased total protein in the cultures as well as stimulation of new DNA synthesis within twenty-four hours of treatment. In the injured defect, protein content increased 35-86% in the presence of the active compounds.

TABLE 2
Effect of Growth Factors on Protein Synthesis in
Wounded Mineralized MC3T3-E1 Cells

| | Control | IGF-1 | PDGF | TGFβ | PGE₂ | FGF-2 |
|---|---|---|---|---|---|---|
| Total | 1.00 | 1.19 | 1.38 | 1.55 | 1.51 | 1.49 |
| Protein/Culture | | | | | | |
| SD | 0.08 | 0.20 | 0.06 | 0.28 | 0.16 | 0.15 |
| P value | | n.s. | 0.0003 | 0.0092 | 0.0013 | 0.0012 |

n.s. = not significant

To supplement the quantitative data on the induction of DNA and protein synthesis by the growth factors in wounded osteoblast cultures, the specimens were studied by epi-fluorescent microscopy with and without treatment 24 hours after injury under the same conditions as described above. The cytoskeletal morphology was visualized using rhodamine phalloidin, an F-actin probe.

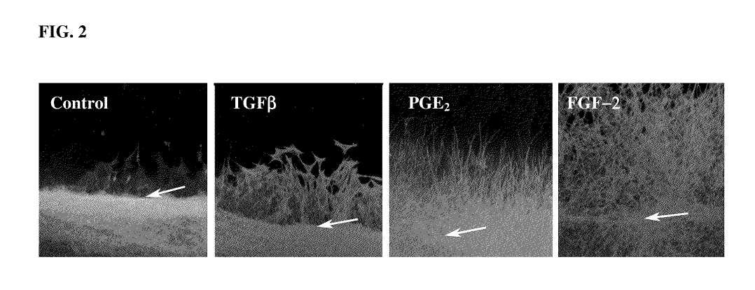

Injured cultures treated with growth factors showed marked morphologic differences at the edge of the defect in the cell layers. In untreated cultures (Control), there was minimal re-growth of cells into the defect created by removal of the cover slip although some re-growth, or cell migration into the defect did occur (FIG. 2). Cultures treated with IGF-1 and PDGF also showed little and unremarkable re-growth and resembled untreated cultures. In contrast, the cultures treated with TGFβ, PGE₂, or FGF-2 (FIG. 2) showed cellular proliferation at the interface of the mature cultures and the defect. Each of these substances produced a distinct pattern in the rhodamine labeled actin cytoskeleton and migration pattern. The relative migration of non-treated defect was 0.95±0.2 mm, while the growth factor TGFβ induced 1.8±0.4 mm migration, PGE₂ induced 3.2±0.2 mm migration, and FGF-2, induced 5.4±0.5 mm migration.

The cells incubated with FGF-2 (0.2 ng/ml) (FIG. 2) showed rapid coverage of the defect void with a random vector growth with formation of a few pseudopodia. The cultures treated with PGE₂ (10 μg/ml) displayed a more linear pattern and formed multiple elongated pseudopodia (FIG. 2).

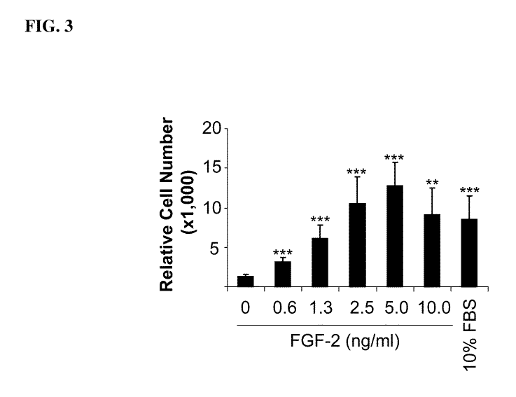

The effect of FGF-2 on proliferation of human mesenchymal stem cells (hMSC) was tested. Relative cell number was obtained with a CyQuant® Cell Proliferation kit. The proliferation dose response for hMSC cells was assessed and the optimum dose was found to be approximately 2.5-5 ng/ml of medium (very similar that that of mouse MSCs) after 4 days of treatment (FIG. 3).

### Example 2

### Effect of Growth Factor Treatment on Gene Expression

This example describes the effect of growth factor treatment of MC3T3-E1 cells and hMSC cells on expression of genes involved in cell proliferation and mineralization.

### Methods

Cell Culture: MC3T3-E1 cells were grown and mineralized essentially as described in Example 1. Mineralizing MC3T3-E1 cells were treated with FGF-2 (5 ng/ml) or BMP-2 (100 ng/ml) for 24 hours. hMSC cells were expanded with 5 ng/ml FGF-2 for 3 to 10 days, then treated with DM for 3 or 10 days.

RNA Isolation: RNA was isolated using the RNeasy™ Mini kit (Qiagen, Valencia, Calif.) or TriReagent™ (Sigma-Aldrich, St. Louis, Mo.) according to the manufacturer's protocol. Cells were seeded in 6-well plates with αMEM medium supplemented with 10% FBS, then down-regulated and activated as indicated.

Reverse Transcription: 1.5 μg of RNA was added to 30 μl reverse transcriptase (RT) reaction buffer containing 5 mM MgCl₂, 10 mM Tris-HCl (pH 8.3), 50 mM KCl, 1 mM dNTPs, 2.5 μM oligo d(T) primer, 2.5 U/μl of MuLV RT, and 1 U/μl of RNase inhibitor. The RT reaction was incubated at room temperature for 10 min, 42° C. for 30 min, inactivated at 99° C. for 5 min, and cooled at 5° C. for 5 min.

Real-time Quantitative RT-PCR Reaction (qRT-PCR): 2 μl of cDNA from the RT reaction was added to 20 μl real-time quantitative polymerase chain reaction (qRT-PCR) mixture containing 10 μl of 2×SYBR® Green PCR Master Mix (Applied Biosystems, Foster City, Calif.) and 12 pmol oligonucleotide primers. PCR was carried out in a Bio-Rad MY iQ® Single-Color Real-Time PCR Detection System (Bio-Rad, Hercules, Calif.) at 50° C. for 2 min, 95° C. for 10 min to activate the Taq polymerase, followed by 50 amplification cycles, consisting of denaturation at 95° C. for 1 min 40 sec, annealing at 63° C. for 1 min 10 sec, and elongation at 72° C. for 1 min 40 sec. Fluorescence was measured and used for quantitative purposes. At the end of the amplification period, melting curve analysis was performed to confirm the specificity of the amplicon. RNA samples were normalized to a cyclophilin (CPHI) internal standard. Relative quantification of gene expression was calculated by using the 2^{-(Ct gene T-Ct CPHI T)-(Ct gene 0 hr-Ct CPHI 0 hr)} equation, where “Cₜ gene T” represents the calculated threshold cycle (Cₜ) of a time point of each sample other than 0 hr, or each treatment other than control. Relative gene abundance is calculated using 2^{(Ct gene T-Ct CPHI T)}, resulting numbers were then multiplied by 10,000 for better graphical presentation. Primer sequences were previously described (Hughes-Fulford and Gilbertson, FASEB J. 13:S121-127, 1999; Hughes-Fulford et al., Gravit. Space Biol. Bull. 11:51-60, 1998; Tjandrawinata and Hughes-Fulford, Adv. Exp. Med. Biol. 407:163-170, 1997; Hughes-Fulford, Adv. Exp. Med. Biol. 400A:269-278, 1997; Hughes-Fulford and Lewis, Exp. Cell Res. 224:103-109, 1996). All data derived using qRT-PCR was from multiple experiments with triplicate independent biological samples.

### Results

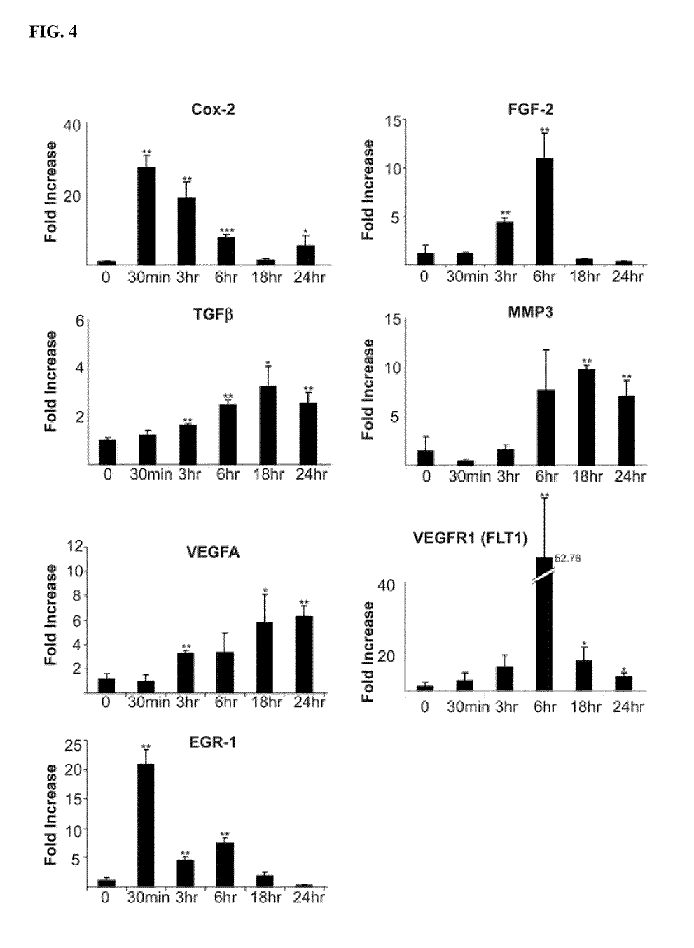

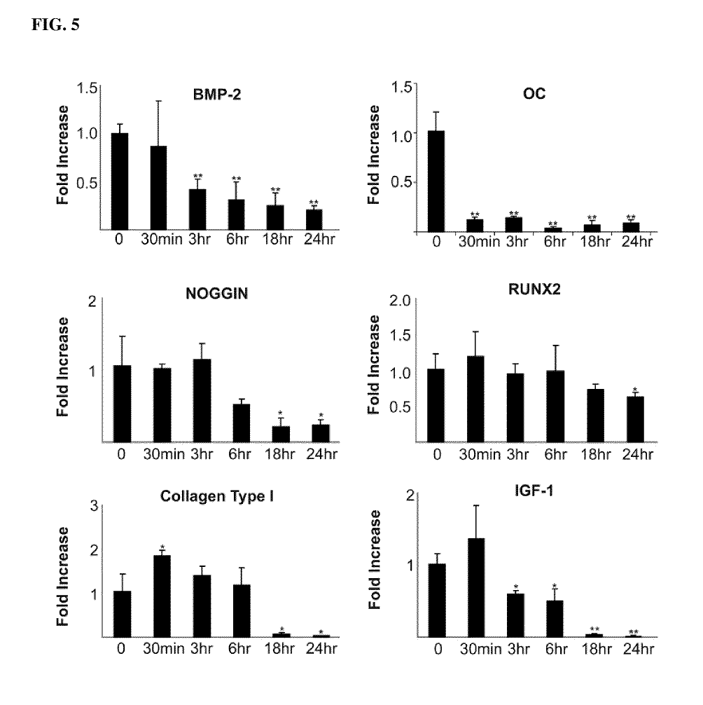

qRT-PCR demonstrated that FGF-2 dramatically induced early growth response-1 (egr-1), fgf-2, cyclooxygenase 2 (cox-2), tgfβ, matrix metalloproteinase 3 (mmp3), vascular endothelial growth factor A (vegfA), and vascular endothelial growth factor receptor-1 (vegfr1) expression in MC3T3-E1 cells over a 24 hour period, with each gene displaying a different sequential temporal pattern of gene induction (FIG. 4). VegfA and vegfr1 are associated with angiogenesis, while mmp3, is associated with increased migration. Tgfβ, fgf-2, egr-1, and cox-2 are key genes in regulation of osteoblast proliferation. Insulin-like growth factor-1 (igf-1), a known differentiation factor, was significantly decreased by FGF-2 treatment (FIG. 5). FGF-2 also significantly decreased expression of genes associated with mineralization including collagen type 1 (col1a1), fibronectin (fn), bmp-2, osteocalcin (oc), runt-related transcription factor type 2 (runx2), and noggin in MC3T3-E1 cells (FIG. 5).

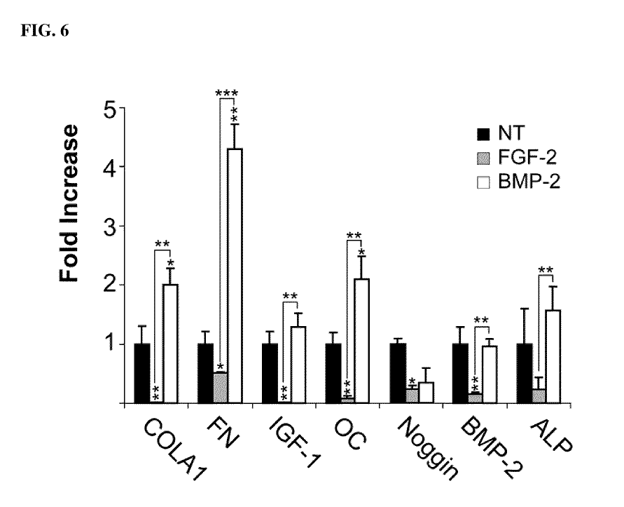

BMP-2, a known promoter of mineralization, was used to study relative abundance of gene expression in mineralizing cells after 24 hours of treatment. As seen in Table 3, BMP-2 treatment caused significant increases in genes associated with mineralization including col1a1, fn, noggin, and oc. Moreover, BMP-2 treatment caused little or no changes in expression of genes associated with angiogenesis and migration e.g. vegf and mmp3. When compared with relative gene abundance of FGF-2 treated cells (FIG. 6), in general, BMP-2 maintained the mineralizing RNA profile of igf-1, alkaline phosphatase (alp), and bmp-2 and significantly increased expression of other genes associated with mineralization, such as col1a1, fn, and oc. FGF-2, on the other hand, significantly suppressed expression of mineralizing genes (Table 3).

TABLE 3
Relative Abundance of Gene Expression in FGF-2 and BMP-2 Treated
MC3T3-E1 Cells

| | | | | | | | FGF-2 vs. |
|---|---|---|---|---|---|---|---|
| | Non-treated | | FGF-2 treated | | BMP-2 treated | | BMP-2 |
| Gene | Avg. | SD | Avg. | SD | Avg. | SD | p-value |
| Collagen | 85081.73 | 25316.39 | **678.21 | 358.27 | *170243.43 | 24493.77 | 0.0003 |
| Type I | | | | | | | |
| Fibronectin | 55827.93 | 12119.18 | *28432.19 | 1195.92 | **239750.67 | 23464.19 | 0.0001 |
| IGF-1 | 3249.51 | 689.70 | **50.65 | 13.30 | 4193.34 | 739.19 | 0.0006 |
| RUNX2 | 349.09 | 40.63 | **674.95 | 63.04 | 1043.65 | 783.29 | n.s. |
| VEGFA | 109.49 | 38.86 | **5132.66 | 755.22 | 537.13 | 379.66 | 0.0007 |
| TGFβ | 93.08 | 10.55 | **245.40 | 41.93 | *185.20 | 38.34 | n.s. |
| ALP | 58.30 | 34.81 | 13.39 | 11.68 | 91.77 | 23.15 | 0.0064 |
| OC | 16.20 | 3.19 | **1.38 | 0.65 | *34.04 | 6.11 | 0.0008 |
| Noggin | 7.11 | 2.77 | *1.61 | 0.49 | 2.41 | 1.76 | n.s. |
| BMP-2 | 0.40 | 0.12 | **0.06 | 0.01 | 0.38 | 0.05 | 0.0004 |
| MMP3 | 0.03 | 0.03 | **4.04 | 0.97 | 0.12 | 0.14 | 0.0023 |

*p < 0.05;
**p < 0.01;
***p < 0.0001 against 0 hour control samples with 2 tail student t test.
n.s., not significant

A hMSC population was expanded with 5 ng/ml FGF-2 for 14 days in DMEM with 10% FBS and then tested for ability to induce osteogenic markers. As early as 3 days of DM treatment, osteogenic markers ALP, RUNX2 and COL1A1 were significantly induced in DM treated cells as compared to controls (Table 4).

TABLE 4
Relative Abundance of Mineralizing Genes in hMSC cells

| | | | DM treated | | DM treated | |
|---|---|---|---|---|---|---|
| | Control | | 3 days | | 10 days | |
| Gene | Avg | SD | Avg | SD | Avg | SD |
| Collagen Type I | 93.5 | 4.5 | 180.2 | 0.02 | 170243.4 | 24493.7 |
| RUNX2 | 20.1 | 2.9 | 31.2 | 0.029 | 1434.6 | 7.8 |
| ALP | 18.3 | 0.6 | 65.4 | 1.68 | 91.7 | 23.1 |

### Example 3

### Effect of Extracellular Matrix and FGF-2 on Cell Growth and Migration

This example describes the effect of FGF-2 and extracellular matrix components on growth and migration of osteoblast or stem cells.

### Methods

Cell Proliferation: Five-hundred MC3T3-E1 osteoblast like cells were plated on a 96 well plate with ECM proteins coated on the bottom of the well. Sextuplet independent samples with and without FGF-2 were each plated in wells and 500 MC3T3-E1 cells were then adhered and grown for 3 days. Cell number was measured using CyQuant assay kit. Medium was changed daily

Cell Migration into Sponge: Collagen sponges (0.5×0.25×1 cm, SURGIFOAM®, Johnson & Johnson, New Brunswick, N.J.) were pretreated with 100 ng/ml FGF-2, 0.1 mg/ml fibrinogen, 0.1 mg/ml fibrinogen with 100 ng/ml FGF-2, 0.3% w/w heparin with 1% hyaluronic acid (HAHp), or HAHp with 100 ng/ml FGF-2. 200,000 MC3T3-E1 osteoblast cells were seeded onto the sponges. Four days after seed up, sponges were evaluated under the microscope to assess cell growth.

Gelatin sponges (GELFOAM®, Pfizer, New York, N.Y.) were pretreated with 100 ng/ml FGF-2, 1 mg/ml fibrinogen, 1 mg/ml fibrinogen with 100 ng/ml FGF-2, 3 mg/ml heparin with 1 mg/ml hyaluronic acid (HAHp), or HAHp with 100 ng/ml FGF-2. 500,000 mouse MSC cells were seeded onto the sponges. Six days after seed up, sponges were evaluated under the microscope to assess cell growth.

Growth on Nylon Ribbons: 250,000 MC3T3-E1 osteoblast cells were seeded in 12-well plates containing 1 inch by 1 inch nylon ribbons pre-treated with 0.1 mg/ml fibrinogen or 1 mg/ml HA and 3 mg/ml heparin (HAHp) with or without 50 ng/ml FGF-2. Cells were visualized under 10× objective at 1 and 4 days after seeding.

### Results

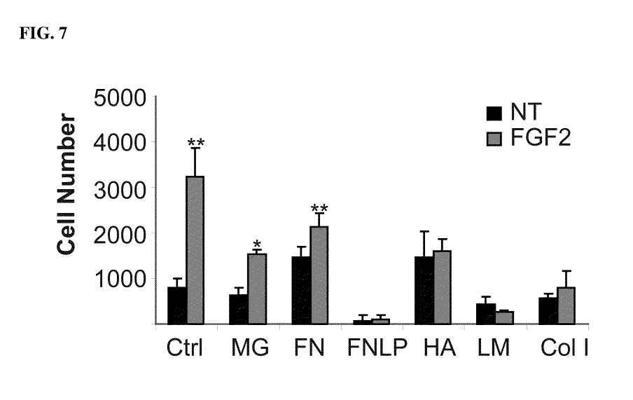

The ability of various ECM proteins and combinations to increase MC3T3-E1 cell proliferation in the presence of FGF-2 was tested. MC3T3-E1 cells were plated in wells containing ECM proteins and FGF-2. MATRIGEL® (a mixture of about 56% laminin, 31% collagen type IV, and 8% entactin; BD Biosciences, San Jose, Calif.), and fibronectin each significantly increased MC3T3-E1 cell proliferation (FIG. 7).

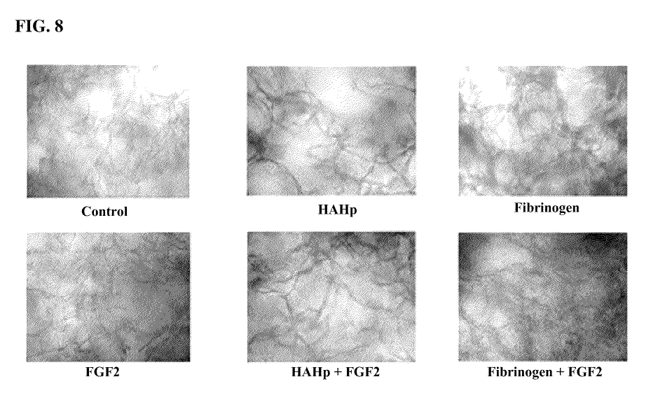

The ability of ECM components and FGF-2 to stimulate migration of human MSCs into a collagen sponge was evaluated. Migration of the cells was assessed by determining cell growth in the center of the sponge. Qualitative determination of cell growth in the center of the sponge is shown in Table 5. Some cells were observed in the sponge pre-treated with FGF-2. Substantially more cells were observed in the sponge pre-treated with fibrinogen plus FGF-2 (FIG. 8).

TABLE 5
Cell Growth in Collagen Sponge with Extracellular Matrix

| Matrix^{a} | Cell Growth in Sponge^{b} |
|---|---|
| Control | − |
| FGF2 | + |
| Fibrinogen | − |
| Fibrinogen + FGF2 | +++ |
| HAHp | − |
| HAHp + FGF2 | − |

^{a}Collagen sponge (0.5 × 0.25 × 1 cm) pre-treated with 100 ng/ml FGF-2; 0.1 mg/ml fibrinogen; 0.1 mg/ml fibrinogen with 100 ng/ml FGF2; 0.3% w/w heparin with 1% hyaluronic acid (HAHp) or HAHp with 100 ng/ml FGF2.
^{b}Cell growth in the center of the sponges was evaluated under the microscope after four days. One positive (+) indicates low cell number visible in the field; two positives (++) indicates a thin layer of cells visible in the field; three positives (+++) indicates many cells visible in the field.

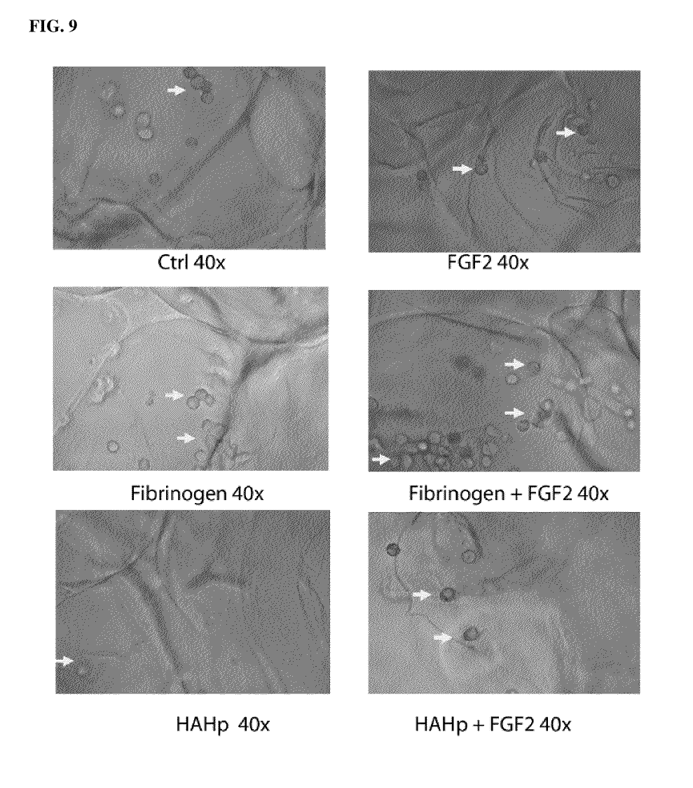

Similarly, the ability of ECM components and FGF-2 to stimulate migration of mouse MSC cells into a gelatin sponge was evaluated. Substantially more cells were observed in the sponge pre-treated with fibrinogen and FGF-2 than control sponges or sponges pre-treated with FGF-2 alone, fibrinogen alone or HAHp with or without FGF-2 (FIG. 9).

Finally, the ability of ECM components and FGF-2 to promote growth of MC3T3-E1 cells on a nylon ribbon was evaluated. Little or no cell proliferation was observed on untreated ribbon or on ribbon pre-treated with FGF-2 alone or fibrinogen alone. Limited cell growth occurred on ribbon pre-treated with HAHp or HAHp and FGF-2. Substantial cell growth was present on the ribbon pre-treated with fibrinogen and FGF-2, reaching near confluency in 4 days. In addition, cells grown on nylon ribbon pre-treated with fibrinogen and FGF-2 formed a cell monolayer, while other pretreatments did not result in formation of full monolayers.

### Example 4

### Mineralization of Osteoblast Cells

This example demonstrates conditions for mineralization of osteoblast cells in vitro.

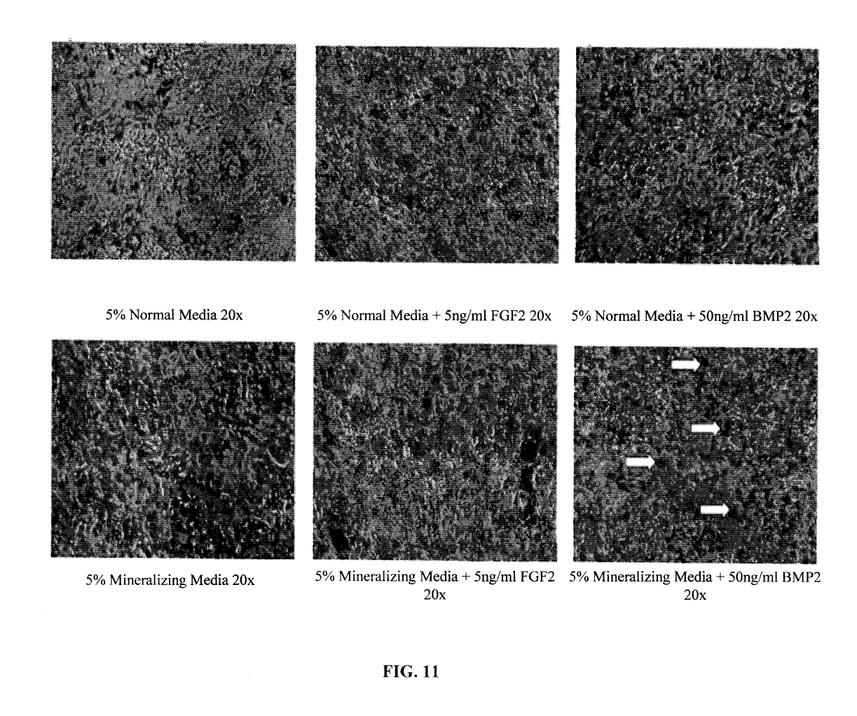

MC3T3-E1 osteoblasts were seeded at 3000 cells/well in 96 well CELLBIND® plates in normal medium (NM; αMEM with 10% FBS, L-glutamate, and antibiotics). Once cells were confluent, media was changed to 5% NM or 5% mineralizing media (MM; NM plus 10 mM β glycerol phosphate and 300 mM L-ascorbic acid) with or without 5 ng/ml FGF-2 or 50 ng/ml BMP-2. Two days after treatment, media was removed and cells were fixed in 10% formalin and stored at 4° C. until subsequent analysis. Cells were stained for calcium with 2% Alizarin Red for 10 minutes and visualized under 20× objectives for photography. Many areas of mineralization, as seen by bright red staining, were present in the cells treated with 5% MM plus 50 ng/ml BMP-2 (FIG. 11). Little to no mineralization was seen with other treatments.

Following photography, mineralization of the cells was quantified as described in Gregory et al., Anal. Biochem. 329:77-84, 2005. Briefly, the Alizarin Red stained cells were incubated with 10% acetic acid for 30 minutes to release bound Alizarin Red into solution. The solution was neutralized with 10% ammonium hydroxide and the absorbance of Alizarin Red was measured at 450 nm using a microplate reader. Data was expressed at in absolute amounts according to a standard curve (Table 6).

TABLE 6
Mineralization of MC3T3-E1 cells in NM or DM

| | Avg. | |
|---|---|---|
| Medium | Conc. | Std. Dev. |
| 5% NM | 5.6 | 1.7 |
| 5% NM plus 5 ng/ml FGF-2 | 5.3 | 0.9 |
| 5% NM plus 50 ng/ml BMP-2 | 16.2 | 4.2 |
| 5% MM | 9.1 | 2.0 |
| 5% MM plus 5 ng/ml FGF-2 | 4.9 | 1.1 |
| 5% MM plus 50 ng/ml BMP-2 | 55.2 | 12.7 |

### Example 5

### Effect of Growth Factors on Cell Migration

This example describes experiments for analyzing the effect of growth factors, such as FGF-2, on cell migration in vitro. However, one skilled in the art will appreciate that methods that deviate from these specific methods can also be used to successfully determine cell migration in response to growth factors.

Migration in defect model: The ability of FGF-2 to induce migration is assessed using the coverslip in vitro defect model as described in Example 1. After cells (MC3T3-E1 cells, mouse or human MSC, or mouse or human ASC) are confluent, they are placed in DM for 7-10 days; once the cells are fully mineralized, the defect is formed by removing the center coverslip. FGF-2 is added to the medium (0.1-1 ng/ml) or ECM/FGF-2 mix as described in Example 3 is added into the center of the defect. After about 20 hours, cells are fixed with 10% formalin and stained with rhodamine phalloidin for F-actin and Hoechst 33258 for nuclei. Images of migrating cells are acquired, for example, using a Zeiss Axiophot microscope and Hamamatsu ORCA digital camera. Migration distance is measured from the cut injury to the tip of the migrating field for example, with AxioVision v4.6 software (Carl Zeiss, NY). Distances are measured to determine average distance in microns±SD.

Migration analysis using migration chamber: FGF-2 induced migration is assessed using the INNOCYTE™ cell migration assay (Calbiochem, San Diego, Calif.). This cell migration assay contains an upper chamber cell culture insert with an 8 μm pore size membrane that cells must pass through to get to the lower chamber. Cells (MC3T3-E1 cells, mouse or human MSC, or mouse or human ASC) are washed and resuspended in serum-free medium, and about 300,000 cells are applied to the upper chamber. The cells are allowed to migrate over a 24-hour period. The bottom chamber contains no treatment, FGF-2, FGF-2 with ECM, ECM alone, or a negative control. Latrunculin A, an inhibitor of actin polymerization, is the negative control for all conditions (3 μM). Migration is determined by fluorescence level (according to manufacturer's protocol), which is proportional to the number of cells adhered on the bottom chamber. Migratory cells migrate through the membrane and attach to the bottom of the polycarbonate membrane that is tissue culture-treated to enhance cell attachment. Migratory cells attached to the bottom of the membrane are stained with calcein-AM and dislodged. The calcein-AM is a cell-permeable dye that is cleaved by intracellular esterases, resulting in fluorescence. Percentage of migratory cells is determined.

### Example 6

### Ectopic Mineralization of FGF-2/BMP-2 Treated Cells on Collagen Sponge

This example describes methods for growth and differentiation of stem cells on a collagen sponge using FGF-2 and BMP-2 treatment and assessment of formation of ectopic mineralized bone in a mouse model following implantation of the sponge. However, one skilled in the art will appreciate that methods that deviate from these specific methods can also be used to grow and differentiate cells and assess mineralization.

Stem cells (mouse or human MSCs) are collected as described in Example 1. Cells are cultured in βMEM with 2% FBS and 0.2-8 ng/ml FGF-2 at 37° C. for 5-10 days. Pieces of collagen sponge (0.5 inches×0.25 inches) are pre-treated with FGF-2 or FGF-2 plus ECM (such as fibronectin, collagen, hyaluronic acid, or fibrinogen). Untreated sponge is used for negative controls. Approximately 2×10⁶ cells are loaded on the sponge, which is then cultured in αMEM with 2% FBS and 50-100 ng/ml BMP-2 at 37° C. for 5-10 days.

Following culture with BMP-2, a small incision is made on the left hind flank of 6-8 week old mice (such as SCID/beige mice) and the sponge is implanted subcutaneously. The sponge is left in place for 1-3 months and mineralization is monitored by periodic μCT scans. The mineralized matrix is analyzed, for example, with a Scanco Medical μCT apparatus (Scanco Medical, Zurich, Switzerland). Three-dimensional (3D) information is obtained by stacking successively measured slices on top of each other. One hundred twenty-eight slices are measured in each sample, covering a total of 1.15 mm of the metaphysis. With a 3D box-shaped low-pass filter applied to the original gray-scale CT images, an artificial partial volume effect is created, which leaves a dense compact shell intact (seen by a smooth surface) and lower lying trabecular type structures visible in the 3D image.

### Example 7

### In Vivo Bone Repair Model

This example describes experiments for testing use of stem cells to repair a bone defect in an in vivo mouse bone fracture model. One skilled in the art will appreciate that methods that deviate from these specific methods can also be used to assess bone repair in a mouse model.

Bone fracture model: A bone fracture is created in SCID/beige mice. Mice are anesthetized with isoflurane using a standard inhalant pump in which oxygen is mixed with isoflurane (2% isoflurane). The surgical area is prepped in standard aseptic fashion. A 2 cm incision on the dorsal aspect of the forelimb is made, exposing both the radius and ulna. The radius is gently dissected free from soft tissue and a 4 mm defect is created at the middle third of the radius with scissors. Care is taken not to injure the adjacent ulna so the animals are able to ambulate. A collagen sponge carrying differentiated stem cells prepared as described in Example 5 is placed in the defect. The fascia and skin are closed separately with 4-0 vicryl. Pain medication is given as needed postoperatively.

Three days before creation of the defect model and 3 days prior to euthanasia, 20 mg/kg of tetracycline (TERRAMYCIN®, 200 mg/ml; Pfizer Animal Health) is injected intraperitoneally to label new bone growth. Radial samples are collected at the time of sacrifice and are fixed in 70% ethanol and embedded in methyl methacrylate for analysis. The samples are sectioned and analyzed by fluorescence microscopy. The distance between the two tetracycline labels represents the extent of new bone formation in over the healing period. In this protocol the 4 mm bone that is removed to form the defect is placed into a tube with PBS to collect any released FGF-2 or BMP-2 from the bone fragment; released growth factors are analyzed with ELISA assays.

μCT scans are conducted every 3 weeks. Once healing is complete or after a pre-determined period of time, the sponge-bone complex at the fracture site is analyzed for mineralization, bone formation, and RNA expression. The sponge-bone complex on the fracture site is decalcified and divided into three parts at the end of the experiment. The first part is used to determine mineralization using alizarin red S. Briefly, samples are washed twice with PBS and fixed for 15 min in 70% ethanol, followed by incubation with 0.5% alizarin red solution for 5 min. Excessive dye is removed by washing 4 times with distilled H₂O. Documentation is made with first color photography, then analyzed calorimetrically. The alizarin red stained samples are incubated with 100 mM cetylpyridinium chloride for 10 min to release calcium bound alizarin red into solution. The absorbance of the released alizarin red is measured at 595 nm using a microplate reader. Data are expressed as arbitrary units alizarin red stain.

The second portion is extracted for RNA analysis. Gene expression of Cox-2, FGF-2, TGFβ, MMP3, VEGFA, VEGFR1, EGR-1, BMP-2, osteocalcin, noggin, RUNX2, collagen type I, IGF-1, osteopontin, and osteonectin are determined by RT-PCR. The third portion of the sponge-bone complex from the radial defects is fixed in 10% buffered formalin, followed by decalcification in 10% EDTA solution for 2 weeks at room temperature with gentle stirring. Sections are cut, stained, and analyzed, facilitating the identification of bone formation. Mineralization formation of trabecular and cortical bone is determined. The analysis of healing is based on mineralization and μCT scan.

Calvarial defect model: Mice are anesthetized with 1.5 mg of ketamine and 0.3 mg of xylazine intraperitoneally. The surgical area is prepped in standard aseptic fashion. A 6 mm defect is made on the calvaria leaving the periosteum intact. A collagen sponge carrying differentiated stem cells prepared as described in Example 5 is placed in the defect. After implantation of the sponge, the skin is closed with 4-0 Ethilon suture. Micro CT of the skull is taken at the time of creation of the defect and at 7, 14, and 21 days. At day 21, mice are sacrificed by cervical dislocation and calvaria are removed, fixed in 10% formalin, decalcified in 10% EDTA, dehydrated in graded alcohols, and embedded in paraffin. Representative sections are cut and stained with hematoxylin and eosin. Bone formation on the calvarial surface is assessed in the lateral aspect of the parietal bone where bone resorption is minimal. Sections spanning a total of 600 μm are obtained from each calvaria, and the width of new bone is quantified from the section with maximal bone formation. Newly synthesized bone is identified in the decalcified calvarial sections by its differential staining with eosin as well as by the woven structure of the collagen when viewed under polarized light. The mean percentage of periosteal new bone area is compared with the total calvaria bone.

### Example 8

### Three-dimensional Culture of Bone Repair System

This example describes exemplary methods for preparing a bone repair system utilizing three-dimensional cell culture. One skilled in the art will appreciate that methods that deviate from these specific methods can also be used.

Stem cells (mouse or human MSCs) are collected and isolated as described in Example 1. Cells are cultured in αMEM with 2% FBS and 0.2 to 8 ng/ml FGF-2 at 37° C. for 5-10 days. Pieces of collagen sponge (0.5 inches×0.25 inches) are pre-treated with FGF-2 or FGF-2 plus ECM (such as fibronectin, collagen, hyaluronic acid, fibrinogen). Untreated sponge is used for negative control. Approximately 2×10⁶ cells are loaded on the sponge, which is then grown in three-dimensional culture in a rotating wall vessel (RWV) or random positioning machine (RPM) for 1-15 days for the cells to populate the sponge. Culture conditions are as described in Example 3. Cell proliferation is measured using CyQuant™ cell proliferation assay or determining DNA content as described in Example 1.

Following cell proliferation in the sponge, 25-100 ng/ml BMP-2 is added to the culture medium and culture is continued in the RWV or RPM. Differentiation of the cells is assessed periodically (such as at days 15, 21, 28, and 35 after start of culture in the sponge) by measuring expression of differentiation markers such as osteocalcin, bone sialoprotein, and collagen type I, as described in Example 2. Mineralization is analyzed by Alizarin Red S assay as described in Example 6.

In view of the many possible embodiments to which the principles of the disclosure may be applied, it should be recognized that the illustrated embodiments are only preferred examples and should not be taken as limiting the scope of the invention. Rather, the scope of the invention is defined by the following claims. We therefore claim as our invention all that comes within the scope and spirit of these claims.

We claim:

1. A method of making a biocompatible transplantable bone repair system, comprising:
culturing multipotent stem cells with a first medium comprising fibroblast growth factor-2 (FGF-2) in the substantial absence of dexamethasone;
seeding a biocompatible substrate comprising fibrinogen with the cultured multipotent stem cells; and
inducing the cultured multipotent stem cells seeded on the biocompatible substrate to differentiate into osteogenic cells by culturing the biocompatible substrate seeded with the cultured multipotent stem cells with a second medium comprising bone morphogenetic protein 2 (BMP-2) in the substantial absence of dexamethasone, thereby making the biocompatible transplantable bone repair system.

2. The method of claim 1, wherein the first medium comprises about 1 mg/ml to about 10 mg/ml FGF-2.

3. The method of claim 1, wherein the biocompatible substrate comprises about 0.1 mg/ml to about 10 mg/ml fibrinogen.

4. The method of claim 1, wherein the second medium comprises about 50 ng/ml to about 100 ng/ml BMP-2.

5. The method of claim 1, wherein the biocompatible substrate is collagen, gelatin, hyaluronic acid, polymers, polymer-hyaluronic acid, polymer-bioactive glass, tricalcium phosphate, hydroxyapatite surfaces, other biologically compatible scaffolds, or a combination of two or more thereof.

6. The method of claim 1, wherein the biocompatible substrate is a sponge, a strip, a scaffold, a gel, a three-dimensional implant, or a combination of two or more thereof.

7. The method of claim 6, wherein the biocompatible substrate is a collagen sponge or a nylon strip.

8. The method of claim 1, wherein the biocompatible substrate further comprises FGF-2.

9. The method of claim 8, wherein the biocompatible substrate comprises about 50 ng/ml to about 100 ng/ml FGF-2.

10. The method of claim 1, wherein the multipotent stem cells comprise stem cells obtained from bone marrow or adipose tissue.

11. A method for treating a bone defect in a subject, comprising:
culturing multipotent stem cells with a first medium comprising fibroblast growth factor-2 (FGF-2) in the substantial absence of dexamethasone;
seeding a biocompatible substrate comprising fibrinogen with the cultured multipotent stem cells;
inducing the cultured multipotent stem cells seeded on the biocompatible substrate to differentiate into osteogenic cells by culturing the biocompatible extracellular matrix seeded with the cultured multipotent stem cells with a second medium comprising bone morphogenetic protein 2 (BMP-2) in the substantial absence of dexamethasone; and
surgically implanting the biocompatible substrate with the differentiated osteogenic cells in the subject at the bone defect site, thereby treating the bone defect.

12. The method of claim 11, wherein the first medium comprises about 1 mg/ml to about 10 mg/ml FGF-2.

13. The method of claim 11, wherein the biocompatible substrate comprises about 0.1 mg/ml to about 10 mg/ml fibrinogen.

14. The method of claim 11, wherein the second medium comprises about 50 ng/ml to about 100 ng/ml BMP-2.

15. The method of claim 11, wherein the biocompatible substrate is collagen, gelatin, hyaluronic acid, polymers, polymer-hyaluronic acid, polymer-bioactive glass, tricalcium phosphate, hydroxyapatite surfaces, other biologically compatible scaffolds, or a combination of two or more thereof.

16. The method of claim 11, wherein the biocompatible substrate is a sponge, a strip, a scaffold, a gel, a three-dimensional implant, or a combination of two or more thereof.

17. The method of claim 16, wherein the biocompatible substrate is a collagen sponge or a nylon strip.

18. The method of claim 11, wherein the biocompatible substrate further comprises FGF-2.

19. The method of claim 18, wherein the biocompatible substrate comprises about 50 ng/ml to about 100 ng/ml FGF-2.

20. The method of claim 11, wherein the multipotent stem cells comprise stem cells obtained from bone marrow or adipose tissue.

21. The method of claim 11, wherein the multipotent stem cells are autologous to the subject.

22. A method of making a biocompatible transplantable bone repair system, comprising:
culturing multipotent stem cells with a first medium comprising about 5 ng/ml fibroblast growth factor-2 (FGF-2) in the substantial absence of dexamethasone;
seeding a biocompatible collagen substrate comprising about 0.1 mg/ml fibrinogen and about 100 ng/ml FGF-2 with the cultured multipotent stem cells; and
inducing the cultured multipotent stem cells seeded on the biocompatible collagen substrate to differentiate into osteogenic cells by culturing the biocompatible collagen substrate seeded with the cultured multipotent stem cells with a second medium comprising about 50 ng/ml bone morphogenetic protein 2 (BMP-2) in the substantial absence of dexamethasone, thereby making the biocompatible transplantable bone repair system.
